(19) United States
(12) Patent Application Publication
(10) Pub. No.: US 2011/0081296 A1
(43) Pub. Date: Apr. 7, 2011

(54) SYSTEMS AND METHODS FOR IDENTIFICATION OF CILIOPATHY THERAPEUTICS

(75) Inventors: MAUREEN M. BARR, HIGHLAND PARK, NJ (US); YOUNG-KYUNG BAE, PASADENE, CA (US)

(73) Assignee: RUTGERS, THE STATE UNIVERSITY OF NEW JERSEY, NEW BRUNSWICK, NJ (US)

(21) Appl. No.: 12/890,596

(22) Filed: Sep. 24, 2010

Related U.S. Application Data
(60) Provisional application No. 61/245,344, filed on Sep. 24, 2009.

(51) Int. Cl. A61K 49/00

(52) U.S. Cl. 424/9.2

(57) ABSTRACT

The invention provides systems and methods for identifying therapeutic targets for treating a disease including a ciliopathy. The invention further provides for drug discovery, and animal model systems related to drug discovery. The invention further relates to therapy of genetic disorders of the cellular cilia or basal bodies.

### CROSS REFERENCE TO RELATED APPLICATIONS

This application claims the benefit of priority of U.S. 61/245,344, filed Sep. 24, 2009, the content of which is incorporated herein in its entirety.

### STATEMENT OF GOVERNMENT FUNDING

The described invention was made with government support under Grants DK059418 and DK074746 awarded by the National Institutes of Health. The government has certain rights in the invention.

### SEQUENCE LISTING

This application incorporates by reference the material on the compact disc labeled “COPY 1”, submitted Dec. 13, 2010, that includes the file entitled “117465.012401_ST25.txt” (1.31 KB) created Nov. 9, 2010.

### FIELD OF THE INVENTION

The invention relates to drug discovery, and animal model systems related to drug discovery. The invention further relates to therapy of genetic disorders of the cellular cilia or basal bodies.

### BACKGROUND

Primary cilium are non-motile sensory organelles present in a single copy on the surface of most growth-arrested or differentiated mammalian cells, and defects in their assembly or function are tightly coupled to many developmental defects, disease and disorders. In normal tissues the primary cilium coordinates a series of signal transduction pathways, including Hedgehog, Wnt, PDGFRα and integrin signaling. In the kidney the primary cilium may function as a mechano-, chemo- and osmosensing unit that probes the extracellular environment and transmits signals to the cell via, for example, polycystins, which depend on ciliary localization for appropriate function. Indeed, hypomorphic mutations in the mouse ift88 (previously called Tg737) gene, which encodes a ciliogenic intraflagellar transport (IFT) protein, result in malformation of primary cilia, and in the collecting ducts of kidney tubules this is accompanied by development of autosomal recessive polycystic kidney disease (PKD). While PKD was one of the first diseases to be linked to dysfunctional primary cilia, defects in this organelle have subsequently been associated with many other phenotypes, including cancer, obesity, diabetes as well as a number of developmental defects. Collectively, these disorders of the cilium are now referred to as the ciliopathies.

### Structure and Diversity of Cilia

Cilia and flagella (the terms are equivalent) are antenna-like organelles that emanate from the surface of many growth-arrested or differentiated eukaryotic cells. They consist of a microtubule (MT)-based axoneme ensheathed by a bilayer lipid membrane that is continuous with the plasma membrane of the cell body, but which contains a distinct subset of receptors and other proteins involved in signaling. The axoneme grows out from the distal end of a modified centriole, the basal body, which provides a template for the formation of the nine-fold symmetry of the ciliary axoneme, and also serves to anchor the axoneme in the cell. Separating the ciliary and plasma membrane compartments is a region known as the ciliary necklace. The ciliary necklace is connected via fibers to the transition zone of the basal body, and these fibers are thought to be part of a ‘ciliary pore complex’ through which only select proteins are allowed to enter the ciliary compartment.

In general, cilia are classified as motile (9+2) or non-motile (9+0), where ‘9’ refers to the number of outer doublet MTs present in the ciliary axoneme and ‘2’ or ‘0’ refers to the number of central MTs present. Motility requires the present of axoneme-associated dynein arms to generate power, and for most motile cilia additional accessory structures, for example radial spokes and central pair projections, are involved in regulating dynein-mediated motility. Some motile cilia, however, contain an extra central pair or lack the central pair entirely. For example, cilia with 9+4, 9+2, and 9+0 axonemes have been observed on the notochordal plate of rabbit embryos and these axonemes all contain dynein arms indicating that they are motile. Consistent with this, 9+0 monocilia on the embryonic mouse node as well as 9+0 cilia on Kuppfer's vesicle in Medaka fish, were observed to perform rotational beating in a manner that generates a directional flow across the cell surface, which is required for establishment of the left-right axis.

In mammals, numerous motile 9+2 cilia are present on the surface epithelial cells lining the airways, brain ventricles, and oviducts. The main function of these 9+2 cilia is to promote the movement of fluids or substances, e.g. airway surface liquid, cerebrospinal fluid, or egg cells, across the epithelial surface; failure to do so may result in airway disease, hydrocephalus, or sterility. A single motile flagellum is present on the mammalian sperm cell, whereas the green alga Chlamydomonas, a commonly used model organism for studying ciliary assembly and function, contains two motile flagella that propel the cell towards or away from a light source. In addition to their motile functions, 9+2 cilia also have important sensory functions, which may in part play a role in regulating motility. However, in terms of cilia-mediated signaling, it is the non-motile 9+0 cilia that have attracted the most attention in recent years.

Non-motile 9+0 cilia, also known as primary cilia, are present on most cells in the mammalian body. Like motile 9+2 cilia, the axoneme of 9+0 cilia consists of nine outer doublet MTs that are nucleated by the basal body, but the central MT pair and structures involved in motility (e.g., dynein arms, radial spokes), are lacking. The primary ciliary membrane is enriched for a number of receptors and ion channels, including platelet-derived growth factor receptor (PDGFR)α, somatostatin receptor 3, serotonin receptor 5, melanin-concentrating hormone receptor 1, polycystins 1 and 2, as well as components of the Hedgehog and Wnt signaling pathways. Therefore the primary cilium is considered to function mainly as a sensory organelle. Some of the best examples of primary cilia that act as sensory organelles are the sensory cilia present in vertebrate olfactory organs and the outer segments of vertebrate photoreceptors. The latter are initially formed from primary cilia during development of the eye and remain connected to the inner segment in adult retina by a short ‘connecting cilium’ that is functionally and structurally equivalent to the transition zone of other types of cilia. The outer segment of photoreceptors turn over at a high rate and therefore large quantities of photo transduction proteins are continuously being transported from the inner to the outer segment, mainly via a process known as intraflagellar transport (IFT), which will be described in more detail below. Defects in IFT impair transport of photo transduction proteins from the inner to the outer segment and lead to degeneration of the outer segments, ultimately resulting in blindness. Likewise, the dysfunction of the cilium on olfactory neurons leads to anosmia and results in their degeneration.

In addition to differentiated cells of olfactory and visual organs, cells of many other organs and tissues in the body (e.g. kidney, liver, pancreas, brain, and oviduct) also display 9+0 primary cilia on their surface when the cells are in growth arrest. While these primary cilia are thought to serve as sensory ‘antennae’ that detect and transmit signals from the surrounding environment to the cell body in order to regulate embryonic development and tissue homeostasis in the adult, there is a growing body of evidence suggesting that primary cilia also play a crucial role in cell cycle control. Since the primary cilium is subtended by the basal body, which is equivalent to one of the mitotic centrioles of the centrosome, a prerequisite for cell cycle entry is disassembly of the primary cilium, a tightly regulated and still not well understood process that appears to involve mitotic kinases such as Aurora and NIMA-related kinases. Consistent with a role for primary cilia in growth control, defective primary cilia were hypothesized to be associated with cancers resulting from abnormal mitogenic signaling or von Hippel-Lindau tumor suppressor signaling.

### Assembly of the Primary Cilium

Assembly of the primary cilium begins in G₁ when Golgi-derived (primary) vesicles attach to the distal end of the older (mother) centriole of the centrosome. As ciliogenesis progresses, axonemal subunits are added directly onto the distal end of the mother centriole, and additional vesicles fuse with the primary vesicles eventually forming a membrane sheath around the nascent ciliary axoneme. In addition, the mother centriole acquires accessory structures and appendages that promote docking and attachment of the mother centriole to the apical plasma membrane of the cell. Following docking of the mother centriole to the apical membrane, the axoneme continues to elongate within the membrane-enclosed compartment by addition of axonemal subunits to the distal end of the growing ciliary axoneme. Because the ciliary compartment lacks the capacity for de novo protein synthesis, axonemal assembly depends on transport of ciliary precursors from the base of the cilium to its distal tip. This transport is carried out by IFT, which is essential for the assembly and maintenance of almost all eukaryotic cilia and flagella.

IFT is a highly conserved process initially discovered in Chlamydomonas as a bi-directional movement of groups of large protein complexes (IFT particles) along the ciliary axoneme. Movement in the anterograde (base to tip) direction is mediated via kinesin-2 motors (Kif3a/Kif3b/KAP complex in vertebrates) whereas movement in the retrograde (tip to base) direction is mediated via cytoplasmic dynein 2. These motors attach to the IFT particles, which in turn are thought to be associated with axonemal cargo proteins entering and leaving the cilium. The IFT particles and motors as well as their cargo proteins accumulate near the site where transition fibers contact the ciliary membrane at the base of the cilium prior to entry into the ciliary compartment, and kinesin-2 then transports IFT particles, cargo proteins, and inactive cytoplasmic dynein 2 to the ciliary tip. At the tip, the IFT particles are remodeled, cargo is presumably unloaded, and kinesin-2 becomes inactive while cytoplasmic dynein 2 becomes active and transports the IFT particles and ciliary turn over products back to the cell body for recycling. The mechanisms that regulate IFT at the ciliary base and tip are not well understood, although some key proteins involved, e.g. MAP kinases and IFT172, have been reported.

The IFT particles are composed of about. 16 different polypeptides, which in Chlamydomonas can be separated biochemically into two distinct complexes termed complex A and B. Almost all of the genes encoding IFT particle polypeptides have been cloned and sequenced, and bioinformatic analyses of IFT polypeptide sequences have revealed that many of them contain motifs and domains known to be involved in transient protein-protein interactions, similar to components of coat protein I (COPI) and clathrin-coated vesicles. Functional studies of individual IFT particle proteins in diverse ciliated organisms have confirmed a requirement for these proteins in ciliary assembly, and have further indicated that components of IFT complex B are associated with anterograde IFT while components of IFT complex A primarily function during retrograde IFT. For example, inactivation of the complex B polypeptide IFT88/Polaris, which is encoded by the ift88 (previously called Tg737) gene, impairs primary cilia formation in the mouse, presumably because ciliary building blocks fail to enter the ciliary compartment via anterograde IFT. In contrast, inactivation of the complex A polypeptide IFT139/THM1 in the mouse leads to the formation of stunted, bulbous cilia, presumably due to defective retrograde IFT resulting in accumulation of IFT particles within the cilium.

Because of their essential role in building the primary cilium, IFT proteins are required for appropriate functioning of a variety of cilia-mediated signaling pathways such as Hedgehog) and PDGFRα signaling. However, there is growing evidence that IFT also plays a more direct role in signaling, both in Chlamydomonas as well as in vertebrates, although the exact mechanisms involved are still somewhat obscure.

### Signaling Pathways Coordinated by the Primary Cilium

In normal tissues the primary cilium coordinates a series of signal transduction pathways, including Hedgehog, Wnt, and PDGFRα pathways as well as functioning as a photo-, mechano- and osmosensing unit that probes and relays information from the extracellular environment into the cell. In many cases, proper signaling is tightly coupled to the correct translocation of receptors and down-stream effector molecules involved in signal transduction to the cilium. Much remains unknown about the mechanisms that control trafficking of signal components into and out of the cilium, although IFT is likely to be involved in several aspects of receptor trafficking. Further, a single primary cilium may contain many different signal transduction systems in order to carry out diverse signaling processes in development and homeostasis of tissues. Therefore it is likely that the composition of signal systems closely reflects the functionality of the cell type in different tissues, i.e., that some ciliary signal systems are tissue specific. This may be particularly relevant when comparing cilia situated deep inside various tissues and organs versus cilia that protrude from the apical surface into the lumen of e.g. tubular structures as seen on epithelial and endothelial cells. Also, during development, the composition of ciliary signal systems may change as part of the dynamic process that controls cell differentiation, allocating different signal systems to different cell types to determine cell fate and function.

Several signal transduction pathways have been shown to be coordinated by the primary cilium to control cellular processes during development and in tissue homeostasis.

### Signaling in Primary Cilia on Epithelial and Endothelial Cells

PKD1 and PKD2 may form a mechanosensory complex that coordinates a flow-sensing response in kidney primary cilia. However, ciliary polycystins may have other functions in relaying this response to control development and homeostasis of the kidney. The channel activity and positioning of PKD2 in the cilium is regulated by Fibrocystin, which is indirectly linked to the N-terminus of PKD2 through Kif3a/b of the Kinesin 2 complex. In addition to their role in Ca2+-signaling, the polycystins contribute to maintaining homeostasis through p53 and JNK and also negatively regulate the JAK/STAT and the mTOR pathways. Alterations in mechanostimulation induces cleavage of PKD1, releasing tuberin and mTOR from their flow-dependent inhibition by PKD1, and allowing the transcription factor STAT6 and co-factor P100 to translocate from the cilium to the nucleus. By itself, the PKD1 C-terminal fragment may influence gene transcription and perhaps modulate Wnt signaling, all processes that promote dedifferentiation and proliferation.

The Nephrocystins are also assumed to exert their function through the primary cilium in renal development and maintenance, although the exact mechanisms remain elusive. More than half of the Nphps (1, 4, 5, 6 and -8) have been localized specifically to the ciliary transition zone at the base of the cilium, suggesting a role in ciliary assembly and/or transport of specific proteins into the ciliary compartments. As such, Nphp-1 and -4 have also been proposed to play a role in axonemal or IFT modeling, whereas Nphp-9/Nek8 is necessary for expression and ciliary positioning of Polycystin-1 and -2. Nphp-3 may also impact ciliary length in mammalian cells, and was demonstrated to interact directly and genetically with Nphp-2/Inversin in the establishment of bilateral asymmetry and promotion of tissue polarity. Another hypothesis proposes the existence of one or more nephrocystin complexes at the ciliary transition zone, in line with the BBSome, which may interact with or be part of a ciliary pore complex. In addition, the activation of the transcription factor ATF4/CREB-2 by Nphp-6/CEP290 indicates that the actions of the nephrocystins are complex and may affect ciliary functions on several levels. Furthermore, studies of Nphp-2 and -3 as well as recent findings with ift88, kif3a, and bbs mutants suggest an important role for the primary cilium in regulating Wnt signaling.

Primary cilia on cholangiocytes that extend from the epithelium into the bile duct lumen also possess a series of receptors and signaling molecules that control tissue homeostasis. These include PKD1, PKD2, Fibrocystin, TRPV4 and G protein-coupled purinergic receptor, P2Y12, that ultimately coordinate mechano-, osmo-, and chemo-sensory functions, which, when defective, cause e.g. cystic and fibrotic liver diseases. Primary cilia on endothelial cells (EC) in blood vessels and in endocardium were proposed to function as shear stress sensors. In cultured human umbilical vein EC, the cilia were found to disassemble in response to laminar shear stress. PKD1 strongly localized to EC cilia in embryonic mouse aorta. In cultures of embryonic ECs, fluid shear stress cleaves PKD1 and ultimately leads to changes in Ca2+ signaling and NO synthesis as well as expression of shear responsive genes such as Krüppel-like factor-2. Consequently, dysfunctional cilia in the cardiovascular system may increase the risk for artherosclerosis and hypertension.

### PDGFRα Signaling in Cycle Control and Migration

Signaling via Platelet-Derived Growth Factors (PDGF) and their receptors (PDGFRs) plays an essential role in cell survival, growth control and cell migration during gastrulation, fetal development and in maintenance of tissues in the adult, with defects causing a range of diseases, including cancer, vascular disorders and fibrosis. Studies have shown that PDGFRα-signaling is coordinated by the primary cilium in mouse embryonic fibroblasts (MEFs), i.e., expression of PDGFRα is up-regulated during growth arrest and targeted to the cilium where PDGF-AA-dependent activation of the receptor and its initial down-stream effectors such as Mek1/2 and Akt occurs. In wild type cells, these signaling events stimulate cell cycle entrance, which is blocked in ift88^{orpk} MEFs that lack the primary cilium. Consequently, PDGFRα signaling through the fibroblast primary cilium may be important in tissue homeostasis while perturbations in this pathway could lead to oncogenesis.

The fibroblast primary cilium may function as a cellular GPS (global positioning system) that coordinates directional migration and PDGFRα-mediated chemotaxis. Using micropipettes to generate a PDGF-AA gradient, wild type growth-arrested MEFs respond immediately to PDGF-AA injection, and migrate uniformly towards the pipette, while ift88^{orpk} MEFs do not respond to PDGF-AA and move around randomly. In in vitro wound healing assays, primary cilia in wild type MEFs orient parallel to one another, perpendicular to the wound and incubation with PDGF-AA increases the migration speed and the directional movement of the cells. In contrast, in ift88^{orpk} cells the migration speed is unaffected by PDGF-AA incubation and cells have decreased directionality. PDGFRα-mediated migration is associated with activation of the ubiquitous plasma membrane Na+/H+ exchanger, NHE1, and inhibition of NHE1 reduces PDGF-AA-mediated cell migration speed and directionality of wt MEFs, whereas this inhibition is markedly reduced in ift88^{orpk} MEFs. These results support the conclusion that the primary cilium represents an alternative mechanism of sensing chemotactic gradients and is part of the positioning machinery that coordinates directed migration in wound healing and developmental processes.

Primary cilia may also directly interact with extracellular matrix (ECM) proteins as the cell moves, transmitting mechanical information from the outside milieu to the cell. In vascular smooth muscle cells (VSMCs) primary cilia that contain PKD1, PKD2 and integrins are critical for cell-ECM interaction and mechanosensing that allow the cilia to project into the ECM and potentially control wound healing. Also, in chondrocytes primary cilia make direct physical contact with ECM components via specific ECM receptors, suggesting that mechanical stimuli may be transmitted through the cilium to control tissue development and to construct a mechanically robust skeletal system. Although speculative at this point, the directional migration of fibroblasts may also be similarly regulated through interactions between the cilium and ECM, and potentially in concert with chemoattractants in embryonic patterning and adult tissue reorganization.

### Wnt Signaling

The primary cilium has been proposed to play a role on Wnt signaling that regulates cell proliferation, cell fate determination and migration. Wnt signaling has been described as a network of interacting, rather than individual pathways. Traditionally, Wnt signals are divided into at least three distinct pathways, all initiated by binding of a ligand of the Wnt family to a 7 transmembrane Frizzled (Fz) receptor. Canonical Wnt signaling involves Dishevelled (Dvl) mediated stabilization of β-catenin, which serves as a transcriptional coactivator and in turn induces cell cycle progression, proliferation, differentiation and growth in addition to migration and regulation of embryonic development. Non-canonical, or β-catenin independent, Wnt signaling pathways act through aPCK, CamK and JNK to control cellular polarity, migration and PCP, necessary for convergence extension during e.g. gastrulation and neurulation.

The primary cilium and basal body have been assigned a role in regulating both the canonical and the noncanonical Wnt signaling pathways due to the ciliary/basal body localization of the PCP proteins Inversin, Vangl-2, and Fat4 in addition to members of the degradation complex, GSK-3β and APC. Vangl-2 appears to interact with Prickle-1 and Inversin both of which can suppress Wnt/β-catenin signaling through Dvl degradation, the latter in concert with another ciliary protein Nephrocystin-3. PCP-like phenotypes have been observed in the inner ear of mice where ift88 has been disrupted, and in Bbs4 deficient mice and zebrafish. Further, loss of primary cilia in the ift88^{TG737Rpw} (previously called ORPK or Tg737^{orpk}) mouse causes a series of abnormalities in the pancreas, including extensive cyst formation in ducts associated with defects in cell cycle control. In the dilated ducts and cysts, the cytosolic localization of β-Catenin is increased and there is an increased expression of Tcf/Lef, which activates transcription of Wnt target genes. These observations support the conclusion that primary cilia are associated with regulation of Wnt signaling in the pancreas.

More recently, two different approaches were used to disrupt cilia/basal body function and demonstrated the importance of these structures for regulation of canonical Wnt signaling RNAi-induced basal body disruption impaired gastrulation in zebrafish due to obstructed convergence extension (CE) movements, and was accompanied by moderately increased canonical signaling. The latter was also the response to shRNA knock-down of either BBS4 or BBS6/MKKS in HEK293 cells, which impaired noncanonical WNT5a's ability to suppress the canonical Wnt3a activity. An identical effect was seen in cells where cilia were ablated by knock-down of KIF3A with shRNA. A corresponding hyper-response to Wnt3a was observed in kif3a or ift88^{Tg737Rpw} mouse embryonic fibroblasts (MEF) using a BATgal canonical Wnt reporter as well as in MEFs and Ofd1-deficient murine embryonic stem cells, albeit not in the absence of Wnt3a. In vivo, there was a general increase in canonical Wnt signaling activity in kif3a mutants, although spatially the activity was normal. The phenocopying of these effects by specific inhibition of the proteasomal subunit RPN10, which associates with BBS4, suggests that proteasomal targeting of β-catenin is a process that requires the basal body. Notably, whereas basal body disruption impaired non-canonical signaling and CE, the noncanonical Wnt5a was still able to induce cytoskeletal rearrangements indicative of PCP (planar cell polarity) equally well in heterozygous and kif3a−/− MEFs, suggesting that repression of the canonical pathway and activation of non-canonical Wnt signal are two independent processes involving the cilium or basal body. Further insights into the connection between cilia/basal body and Wnt signaling have been revealed by Bergmann and colleagues who demonstrated that the ciliary protein Nphp3 binds to Inversin and can inhibit Inversin mediated canonical Wnt signaling (Bergmann C, Fliegauf M, Bruchle N O, Frank V, Olbrich H, Kirschner J, Schermer B, Schmedding I, Kispert A, Kranzlin B, Nurnberg G, Becker C, Grimm T, Girschick G, Lynch S A, et al. Loss of nephrocystin-3 function can cause embryonic lethality, Meckel-Gruber-like syndrome, situs inversus, and renal-hepatic-pancreatic dysplasia. Am J Hum Genet 2008; 82:959-970. [PubMed: 18371931]).

### Hedgehog Signaling

Another critical signal transduction pathway that is coordinated by the primary cilium is the hedgehog (Hh) pathway. In additional to its general roles in tissue homeostasis, this pathway is crucial in tissue differentiation during embryonic development, and dysfunction of the Hh pathway is responsible for e.g. basal cell carcinoma, the most common form of cancer in humans.

The hedgehog ligand exists in three different varieties (sonic (Shh), indian (Ihh) and desert (Dhh)) that are spatially and temporally regulated and whose concentration gradient ultimately helps determine the eventual cell fate and proliferation rate. The ligands work through two transmembrane proteins, Patched (Ptc) and Smoothened (Smo) that in turn regulate the activity of three transcription factors of the Gli family (Gli1,2,3). Ptc is the receptor for Hh ligands, and, in the absence of Hh ligands, it negatively regulates Hh signaling by suppressing the activity of Smo. Upon binding of Hh ligand to Ptc, the inhibition of Smo is relieved, preventing processing of Gli3 to a repressor and activating the Gli-2 transcription factor, which in turn induces the hedgehog pathway through their nuclear transcriptional targets.

A series of observations have now shown that primary cilia are critical regulators of the Hh pathway where the regulated concerted movement of Ptc out of and Smo into the cilium may create a switch by which cells can turn Hh signaling on and off during development and in control of tissue homeostasis. In this scenario, binding of ligands to Ptc in the cilium activate the Hh pathway by removal of Ptc from the cilium in a process that is associated with ciliary enrichment of Smo. In vitro activation of Smo in cells exposed to Shh is shown to be blocked in MEFs lacking IFT172 or the dynein retrograde motor, Dync2h1. The translocation of Smo into the primary cilium upon Shh stimulation can also be blocked by knocking down Kif3A or β-arrestins, which are thought to be adapter proteins for the Smo protein.

Mutations in IFT proteins required for ciliary assembly results in dysfunctional Hh signaling and severe developmental disorders in mammals. Removal of Kif3A causes aberrant hedgehog signaling which has been shown to have an impact on skeletogenesis, neural tube formation, and cerebellar development. Mutations in the IFT139 homologue (Thm1) specifically results in abnormal Gli3 activator/repressor ratios which in turn results in defects in the neural tube formation. Similarly, mutations in a basal body protein (Ftm1) also resulted in abnormal ratios of Gli3 activator/repressor which lead to defects in left-right symmetry, neural tube formation and limb development. The inhibition of Gli3 cleavage for subsequent activation of the Hh pathway was shown also to require other ciliary proteins: the retrograde IFT dynein motor subunit, Dnchc2, the IFT172 protein and the ciliary Arl13b (a small GTPase of the Arf/Arl family). Studies have reported that a siRNA screen identified different mediators of the Hh pathway, among them genes controlling ciliogeneis: Nek1 (NIMA-like kinase) and Prka (a kinase participating in miRNA processing and thought to localize at the base of the cilium). Further, deletion of ift88 in ovary using Cre-Lox recombination in mice resulted in a severe delay in mammary gland development and defects in ovarian function, and in IFT57-deficient mice, there were defects in ventral neural tube formation (exencephaly) due to aberrant Shh signaling. These findings highlight the importance of IFT proteins in the hedgehog pathway.

One example of the in vivo changes in the levels of Hh molecules in the cilium comes from studies on the development of the pancreas, which is controlled by the graded response to Hh signaling. Interestingly, Smo and Gli2 are absent from pancreatic primary cilia at human embryonic stage week 7.5, i.e. before formation of the endocrine system, but highly concentrated in cilia in 14 and 18 week old fetuses. This increase in ciliary localization of Smo and Gli2 is accompanied by loss of Gli3 ductal epithelium, suggesting that a graded Hh signaling response coordinated by the primary cilium regulates the development of the human pancreas. Therefore, disruption of pancreatic development in mice with defects in primary ciliary may be due to loss of both coordinated Hh and Wnt signaling during genesis of the pancreas. The canonical Wnt and Ihh pathways may also help to coordinate osteoblast and chondrocyte differentiation during bone development. One known disorder resulting from developmental skeletal problems is chrondoectodermal dysplasia in Ellis-van Creveld syndrome, which arises from mutations in Evc, which localizes to the base of the chondrocyte primary cilium.

Ciliopathies

Hepatorenal fibrocystic diseases (HRFCDs) are among the most common inherited human disorders. The discovery that proteins defective in the autosomal dominant and recessive polycystic kidney diseases (ADPKD and ARPKD) localize to the primary cilia and the recognition of the role these organelles play in the pathogenesis of HRFCDs led to the term “ciliopathies.”

While ADPKD and ARPKD are the most common ciliopathies associated with both liver and kidney disease, variable degrees of renal and/or hepatic involvement occur in many other ciliopathies, including Joubert, Bardet-Biedl, Meckel-Gruber, and oral-facial-digital syndromes. The ductal plate malformation (DPM), a developmental abnormality of the portobiliary system, is the basis of the liver disease in ciliopathies that manifest congenital hepatic fibrosis (CHF), Caroli syndrome (CS), and polycystic liver disease (PLD). Hepatocellular function remains relatively preserved in ciliopathy-associated liver diseases. The major morbidity associated with CHF is portal hypertension (PH), often leading to esophageal varices and hypersplenism. In addition, CD predisposes to recurrent cholangitis. PLD is not typically associated with PH, but may result in complications due to mass effects. The kidney pathology in ciliopathies ranges from nonfunctional cystic dysplastic kidneys to an isolated urinary concentration defect; the disorders contributing to this pathology, in addition to ADPKD and ARPKD, include nephronophithisis (NPHP), glomerulocystic kidney disease and medullary sponge kidneys. Decreased urinary concentration ability, resulting in polyuria and polydypsia, is the first and most common renal symptom in ciliopathies. While the majority of ADPKD, ARPKD, and NPHP patients require renal transplantation, the frequency and rate of progression to renal failure varies considerably in other ciliopathies.

Ciliopathies: Liver Diseases

Intact cilia-based signaling is required for normal development of the biliary and portal system in the liver. The majority of diseases manifesting with hepatic fibrocystic pathology are caused by defective ciliary proteins; exceptions include autosomal dominant polycystic liver disease (ADPLD) and portal fibrosis associated with Congenital Disorder of Glycosylation (CDG) type Ib.

Hepatic fibrocystic pathologies can be grouped into three major descriptive categories: CHF; CD; and PLD. CHF is a histopathological diagnosis with three main components: defective remodeling of the ductal plate (DPM); abnormal portal veins; and progressive fibrosis of the portal tracks. The major morbidity associated with CHF is portal hypertension (PH). CD refers to macroscopic saccular or fusiform dilations of the medium and large sized intrahepatic bile ducts on imaging of the biliary system. Caroli syndrome (CS) refers to CD in association with CHF. CD and CS probably represent different presentations of a continuum of pathology; CD and CS are reported to affect different members of the same family. The liver cysts in CS are non-obstructive dilatations of the intrahepatic biliary tree; hence, they are continuous with the biliary system. In contrast, the liver cysts in PLD are isolated, closed cysts that originate from biliary microhamartomas (von Meyenburg complexes) embedded in fibrous tissue; they are not in continuity with the intrahepatic biliary tree.

DPM (ductal plate malformation) is the main pathology underlying the liver disease in ciliopathies. The severity of DPM and the level of the portobiliary tree affected by DPM vary within and among individual ciliopathies. DPM results in a spectrum of abnormalities including CHF (microscopic bile ducts). CHF/CS (microscopic and medium size bile ducts), and CD (medium and large bile ducts). The isolated liver cysts in the PLD of ADPKD probably represent DPM affecting the most peripheral end of the biliary system.

### Congenital Hepatic Fibrosis/Caroli Syndrome

Typically, CHF/CS occurs as part of a multisystem disorder usually associated with fibrocystic renal disease (Table I). CHF/CS rarely appears as an isolated finding, but no causative gene has been identified to date. CHF/CS most commonly presents as part of autosomal recessive polycystic kidney disease (ARPKD). Although all patients with ARPKD have CHF on microscopic examination at birth, abnormal liver echogenicity and splenomegaly may not be detectable during early childhood because periportal fibrosis and PH are time-dependent pathologies that progress with age. The majority of patients with the CHF associated with ARPKD develop PH (portal hypertension), which results in splenomegaly, esophageal varices and thrombocytopenia and leukopenia due to hypersplenism. The hepatocellular function of the liver is relatively well preserved, and liver enzymes are normal or mildly elevated. An increased risk for cholangiocarcinoma in adulthood and for cholangitis at any age are the major morbidities caused by Caroli syndrome.

Other disorders in which CHF is a universal finding include Meckel-Gruber syndrome (MKS), COACH syndrome (a subset of Joubert syndrome and related disorders (JSRD) with cerebellar vermis hypoplasia, oligophrenia, ataxia, coloboma, and hepatic fibrosis), and renal-hepatic-pancreatic dysplasia (RHPD) (Table I). Symptomatic CHF/CS, associated with PH or cholangitis, occurs in a subset of patients with various other ciliopathies including JSRDs, Bardet-Biedl (BBS), oral-facial-digital (OFD) syndromes; and ciliary skeletal dysplasias such as Jeune asphyxiating thoracic dystrophy (Table I). In most cases, the inheritance pattern of CHF/CS is autosomal recessive. Two rare exceptions are X-linked CHF/CS in association with oral-facial-digital syndrome type I (OFD1) and autosomal dominant CHF/CS in rare families with ADPKD. Whether the microscopic pathology of CHF is a universal finding in these syndromes remains to be determined. Asymptomatic or mild to moderate CHF/CS might be under-diagnosed in these disorders, because the pathologies of other systems often become the focus of attention and lifespan can be limited.

Ultrasonography (USG) is the most informative imaging modality in diagnosing CHF/CS; in patients with CS, it reveals increased echogenicity of the liver with a coarse, nonhomogeneous pattern, splenic enlargement as an evidence for PH, and macroscopic liver cysts. These findings, especially in the context of fibrocystic renal involvement, are diagnostic of CHF/CS; liver biopsy is not required in such cases. However, liver biopsy is often performed in HRFCD patients who present in late childhood or adulthood with splenomegaly or thrombocytopenia (caused by hypersplenism). Liver biopsy in CHF shows abnormal portal tracts with an excess number of abnormally shaped embryonic bile ducts retained in their primitive ductal plate configuration (incorrectly referred to as bile duct proliferation), an abnormal portal vein, and periportal fibrosis without inflammation. Portal-portal bridging of the fibrotic bands is common in advanced CHF; absence of portal tract to central vein bridging and preserved hepatocellular function distinguishes CHF from cirrhosis.

TABLE 1
Ciliary Disorders Associated With Congenital Hepatic Fibrosis/Caroli's Syndrome

| | Mode of | | |
|---|---|---|---|
| Disease | Inheritance | Gene(s) | Prevalence |
| Autosomal recessive | AR | PKHD1 | ~1 in 20,00 |
| polycystic kidney disease | | | |
| Meckel-Gruber syndrome | AR | MKS1, TMEM67, CEP290, | ~1 in 140,000 |
| | | RPGRIP1L, CC2D2A | |
| Joubert syndrome and | AR | AHI1, NPHP1, CEP290, | ~1 in 100,000 |
| related disorders including | | TMEM67, RPGRIP1L, | |
| COACH syndrome | | ARL13B, CC2D2A | |
| Bardet-Biedhl syndrome | AR | BBS1, BBS2, ARL6, BBS4, | ~1 in 100,000 |
| | | BBS5, MKKS, BBS7, TTC8, | |
| | | BBS9, BBS10, TRIM32, | |
| | | BBS12, MKS1, CEP290 | |
| Oral-facial-digital | X-linked | OFD1 | ~1 in 100,000 |
| syndrome Type I | | | |
| Renal-hepatic-pancreatic | AR | NPHP3 | Rare |
| dysplasia | | | |
| Jeune chondrodysplasia | AR | IFT80 | Rare |
| Cranioectodermal dysplasia | AR | Unknown | Rare |
| Ellis-Van Crevald | AR | EVC, EVC2 | Rare |
| syndrome | | | |
| Mainzer-Saldino syndrome | AR | Unknown | Rare |
| Glomerulocystic kidney | AR, AD | HNF-1b | Rare |
| disease | | | |
| Autosomal dominant | AD | PKD1, PKD2 | ~1 in 1,000 |
| polycystic kidney disease | | | |
| Nephronophthisis | AR | NPHP1, INVS, NPHP3, | ~1 in 100,000 |
| | | NPHP4, IQCB1, CEP290, | |
| | | GLIS2, RPGRIP1L, NEK8 | |

### Polycystic Liver Disease (PLD)

PLD refers to the presence of multiple macroscopic cysts in the liver. The cysts are isolated and are not part of the biliary tree. Most commonly, PLD exists as part of ADPKD. Otherwise, PLD occurs in patients with autosomal dominant polycystic liver disease (ADPLD), an entity that is genetically distinct from ADPKD and not typically associated with renal cysts. ADPLD is genetically heterogeneous; the PRKCSH and SEC63 genes, encoding the proteins hepatocystin and Sec63, account for less than half of ADPLD patients. Unlike other cystoproteins, hepatocystin and Sec63 are not ciliary proteins; they are involved in the ER processing of proteins.

### PLD as a Part of ADPKD

ADPKD represents the most common form of HRFCD, with a frequency of 1 in 500-1,000, and PLD is the most common extra-renal manifestation of ADPKD. Liver cysts in ADPKD increase in number and size as patients grow older; 58% of 15- to 24-yearolds and 94% of 35- to 46-year olds have liver cysts. Although the prevalence of liver cysts is comparable in men (79%) and women (85%), cyst volume is greater in women, especially after multiple pregnancies and use of oral contraceptives. Enlarged cysts can cause chronic upper abdominal pain and distention, early satiety, nausea and dyspnea due to a mass effect, but hepatic function remains normal. Cyst infection and hemorrhage can occur. CHF complicated by PH has been described in rare ADPKD families, and CS has also been reported. PLD develops in ADPKD due to both PKD1 and PKD2 mutations, but no genotype-phenotype correlation exists for PLD associated with ADPKD.

Ciliopathies: Kidney Diseases

In ciliopathies, the kidneys are the most commonly affected organ, displaying pathologies ranging from a urinary concentration defect in normal appearing kidneys to cystic dysplastic kidneys. While ADPKD and ARPKD represent the most common ciliopathies, nephronophthisis, cystic dysplastic kidneys, medullary sponge kidney, and several overlap phenotypes contribute to the spectrum of kidney diseases observed in ciliopathies.

Bilateral, diffusely echogenic and/or cystic kidneys on prenatal or postnatal ultrasound are commonly the presenting findings in ciliopathy patients. In healthy infants, the renal cortex is more echogenic than the liver, but this physiological hyperechogenicity of the cortex disappears by about 3 months. In ciliopathies, the kidneys are termed “echogenic” “hyperechogenic” “hyperechoic” or “bright”, describing abnormally increased renal signal density on USG, that is, greater than that of the liver. In addition to ciliopathies, the broad differential diagnosis for hyperechoic kidneys includes congenital infections, renal vein thrombosis and congenital nephrotic syndrome. Determination of the presence or absence of congenital abnormalities other than hepatorenal disease, such as polydactyly, mid and hindbrain defects, iris or retinal colobomas, and retinal degeneration, helps to focus the diagnostic possibilities. The differential diagnosis for bilateral diffusely echogenic and/or cystic kidneys in a non-dysmorphic infant or child without extra-hepatorenal involvement includes ARPKD, ADPKD, glomerulocystic kidney disease (GCKD), and tuberous sclerosis. Although renal cysts in tuberous sclerosis are typically associated with angiomyolipomas, cysts can occur without angiomyolipomas during the first years of life. The appearance of kidneys on USG can be similar in infants with ARPKD, perinatal-onset ADPKD, glomerulocystic kidney disease, cystic dysplastic kidneys (as in classical Meckel-Gruber syndrome), BBS-related kidney disease, MKS-3-related kidney disease and some forms of NPHP. In all cases, the kidneys are enlarged and diffusely hyperechogenic with loss of corticomedullary differentiation, with or without macrocysts (visible discrete cysts).

### Autosomal Dominant Polycystic Kidney Disease (ADPKD)

ADPKD is a systemic ciliopathy in which the kidneys and liver are primarily affected, but cysts can occur in the pancreas, arachnoid membrane, and seminal vesicles. Other manifestations include intracranial aneurysms, dilatation of the aortic root, dissection of the thoracic aorta, and mitral valve prolapse. The majority of ADPKD patients have a positive family history; 5-10% of patients have their disease due to de novo mutations. ADPKD is genetically heterogeneous, with two causative genes identified. PKD1 encoding Polycystin-1 accounts for approximately 85% of affected individuals; PKD2 encoding Polycystin-2 is mutated in the remaining 15% of patients.

Although PKD1 and PKD2 sequencing is available, the diagnosis of ADPKD is made clinically, primarily based on imaging studies. The mutation detection rates for PKD1 and PKD2 are similar at approximately 85-90%. PKD1 and PKD2 gene mutations result in similar extra-renal manifestations, including PLD and intracranial aneurysms, but PKD1 mutations cause more severe kidney disease associated with a 20-year earlier onset of end stage renal disease (ESRD) compared with PKD2 mutations (54.3 years for PKD1; 74.0 years for PKD2). Mutations at the 5′ end of PKD1 are more likely to be associated with intracranial aneurysms and early renal failure than are 3′ mutations.

Although all cells in an ADPKD kidney carry the germline mutation, cysts originate only from a small subset (˜5%) of cells that acquire a second hit. Most ADPKD patients are born with normal kidneys, and cysts develop and increase in number and size over a lifetime; glomerular function typically begins to decline in adulthood after massive cystic enlargement of the kidneys. Decreased urinary concentrating ability and hypertension occur before the start of the decline in glomerular function. ADPKD cysts originate from any part of the nephron and become isolated cysts early in the course of the disease. Early in ADPKD, kidney USG reveals multiple cysts usually in association with normal parenchyma; in children cystic involvement may be unilateral. Rarely, ADPKD may present perinatally with enlarged hyperechogenic kidneys, indistinguishable from ARPKD kidneys on imaging. After the cysts form, they continue to grow into large macrocysts. Consequently, imaging in adults with ADPKD reveals massively enlarged kidneys full of macrocysts and often distorting the contour of the kidneys. Among individuals at 50% risk for ADPKD, diagnostic sensitivity of USG at age 30 years and younger is 95% for PKD1-related ADPKD and 67% for PKD2-related ADPKD. A diagnosis of ADPKD cannot be excluded by a negative USG until the individual is 35 years old. Differing from ARPKD, ADPKD patients suffer from renal pain caused by nephrolithiasis, cyst hemorrhage or infection.

### Autosomal Recessive Polycystic Kidney Disease (ARPKD)

ARPKD, the most common childhood-onset ciliopathy, occurs with a frequence of approximately 1 in 20,000 live births, making the carrier frequency approximately 1 in 70. ARPKD patients have non-obstructive fusiform dilatations of the renal collecting ducts, leading to progressive renal insufficiency, and DPM of the liver, resulting in CHF/CS. Most ARPKD patients present perinatally with enlarged kidneys, and approximately 30% die of pulmonary hypoplasia. CHF invariably accompanies ARPKD; although mild in early childhood, it eventually leads to PH, esophageal varices and hypersplenism in most cases. Severe, early-onset systemic hypertension is seen in approximately 80% of ARPKD patients. It typically requires multi-agent antihypertensive treatment in infancy and becomes relatively easier to control later in childhood. Some ARPKD patients present late in childhood or adulthood, usually with PH associated with milder kidney disease. The majority of ARPKD patients who become symptomatic perinatally require kidney transplantation in late childhood to adulthood. The diagnosis of ARPKD/CHF relies upon clinical, radiographic or biopsy evidence of typical renal pathology and CHF with autosomal recessive inheritance.

Differing from ADPKD, ARPKD kidneys are already diffusely affected at birth. They are symmetrically enlarged due to dilated collecting ducts, and retain their reniform configuration. Although most dilated collecting ducts in ARPKD continue to have urine flow in their lumen, some dilated ducts become closed cysts as children with ARPKD grow. This results in the appearance of macrocysts on the background of diffusely increased echogenicity on USG. As a consequence, imaging findings of ARPKD patients who present in childhood or adulthood can be difficult to distinguish from early ADPKD kidneys.

All patients with ARPKD thus far ascertained, including those who present in childhood or in adulthood with liver-predominant symptoms, have had mutations identified in the PKHD1 gene that encodes fibrocystin/polyductin. Truncating PKHD1 mutations are associated with a more severe phenotype; typically, ARPKD patients who survive the neonatal period have at least one missense mutation.

### Nephronophthisis (NPHP)

NPHP consists of a group of autosomal recessive tubulointerstitial disorders that initially present with a urinary concentrating defect and anemia and subsequently require renal transplant, usually in childhood. Fibrotic tubulointerstitial pathology predominates in NPHP; renal cysts, typically located at the corticomedullary junction, form secondarily. The typical NPHP biopsy shows thickening and disruption of the tubular basement membrane, tubular atrophy, disproportionate tubulointerstitial fibrosis with minimal inflammation, and macrocysts at the corticomedullary junction. Infantile, juvenile, and adolescent forms of NPHP result in ESRD at median ages of 1, 13, and 19 years, respectively. Juvenile NPHP, the most common form, generally presents between ages 4 and 6 years with polyuria, polydipsia, and anemia. Blood pressure is typically normal before the onset of renal failure. Renal USG shows normal size or small kidneys with increased echogenicity, with or without corticomedullary cysts. In infantile NPHP, the kidney size may be enlarged.

A subset of individuals with NPHP have mild retinal degeneration (tapetoretinal degeneration), which is most often asymptomatic. Other extra-renal manifestations include CHF, structural cerebellar and midbrain abnormalities overlapping with JSRD, and severe retinal degeneration (Senior-Løken syndrome). Studies report that up to nine genes (NPHP1-9) have been shown to cause NPHP, but those nine genes account for only 30% of NPHP patients. NPHP1 mutations are the most common cause of NPHP (21%), and each of the other known genes is responsible for 3% or less of the disease. Clinical symptoms and renal histology are similar in all forms of NPHP except for NPHP2, which causes infantile NPHP. CHF is associated with NPHP2 and NPHP3 mutations. It remains to be determined whether other NPHP genes are associated with CHF. MKS3 mutations can cause NPHP with CHF. In addition, some patients with MKS3 mutations display an overlap phenotype with features of both NPHP and ARPKD. The renal disease in Jeune asphyxiating thoracic dystrophy displays features most consistent with nephronophtisis.

### Kidney Disease in Bardet-Biedhl Syndrome

Renal disease is a major cause of morbidity and mortality in BBS. Polyuria and polydypsia due to a urinary concentration defect occur frequently. On ultrasound imaging, BBS kidneys exhibit findings ranging from retained fetal lobulation to enlarged kidneys, resembling those in ARPKD, with diffuse hyperechogenicity and loss of corticomedullary differentiation, with or without macrocysts. On intravenous contrast imaging such as intravenous pyelography, BBS kidneys show renal calyceal clubbing, blunting, and distortion in the absence of distal obstruction. Glomerular function declines at variable rates, requiring renal replacement therapy in some patients. Biopsy of BBS kidneys with declining glomerular function reveals pathology resembling NPHP tubulointerstitial disease.

### Glomerulocystic Kidney Disease (GCKD)

Autosomal dominantly inherited familial hypoplastic GCKD is a distinct form of the disease characterized by small kidneys and medullocalyceal abnormalities; affected individuals have heterozygous mutations in the hepatocyte nuclear factor-1-beta gene (HNF1β or TCF2 mutations in HNF1β also result in maturity-onset diabetes of the young, type V (MODY V, also referred to as renal cysts and diabetes syndrome), which can display a spectrum of renal anomalies ranging from glomerular cysts to renal agenesis. Familial and sporadic GCKD may be associated with renal medullary dysplasia and ductal plate malformation of the liver.

The term glomerulocystic kidney (GCK), which differs from GCKD, refers to kidneys in the context of malformation syndromes such as oral-facial-digital syndrome-type I and trisomies 13 and 18; in these disorders, glomerular cysts are part of the kidney pathology.

Histopathologically, GCKD is characterized by a predominance of glomerular cysts without tubular dilatation. On USG, GCKD kidneys might be small, normal in size, or enlarged with small (<1 cm) cysts in the echogenic renal cortex but not in the medulla. Most GCKD is inherited in an autosomal dominant fashion, but it is not due to PKD1 or PKD2 mutations. In autosomal dominant GCKD, kidneys are bilaterally enlarged and diffusely cystic. Infants with GCKD can have presentations that mimic ARPKD, and adults with GCKD can present with flank pain, hematuria, and hypertension.

### Cystic Dysplastic Kidneys (CDK)

Cystic dysplastic kidneys, which are distinct from multicystic dysplastic kidneys, are characterized by poorly differentiated and disorganized nephron segments with primitive elements such as cartilage. CDKs are better differentiated than multicystic dysplastic kidneys, and have some normal nephrons. Similarly, the collecting system of cystic dysplastic kidneys can by hypoplastic or malformed, but is generally better developed than that of MCDKs. USG of cystic dysplastic kidneys shows echogenic kidneys that are often large, without any demarcation between medulla and cortex. Ciliopathies associated with cystic dysplastic kidneys include Meckel-Gruber and Dekaban-Arima syndromes and renal-hepatic-pancreatic dysplasia.

### Multicystic Dysplastic Kidneys (MCDK)

In MCDK, the kidneys are non-reniform and so grotesquely cystic that they are described as “a bunch of grapes.” The calyceal system, ureter, and renal vasculature are usually atretic. There is no differentiated renal tissue; the non-cystic tissue shows nests of cartilage and mesenchymal mantles surrounding primitive tubules. Thus, MCDKs are always non-functional and usually unilateral, since bilateral MCDK is incompatible with life. USG reveals no identifiable renal parenchyma, but rather several small single cysts or a large mass of cysts of varying sizes surrounding a dominant large cyst. MCDK can occur in MODY 5, a disorder caused by heterozygous mutations in HNF1B; this transcription factor regulates expression of the ARPKD gene PKHD1. MCDK can rarely be familial.

### Medullary Sponge Kidney (MSK)

Most cases of MSK are sporadic; autosomal dominant inheritance is suggested in some Families. MSK is typically diagnosed based on intravenous urography imaging that shows characteristic radial linear streaking in the renal papillae due to collection of contrast medium in the ectatic papillary-collecting ducts; this appearance is often referred to as a “bouquet of flowers.” In approximately 50% of the patients, MSK may be complicated by nephrocalcinosis. MSK is usually bilateral; kidneys are normal size and glomerular function is preserved. Patients usually remain asymptomatic in childhood and come to medical attention in adulthood due to renal lithiasis.

Studies have focused on the nephronal disorders arising from ciliary dysfunction, such as Polycystic kidney disease (PKD), due to which a connection between ciliary malfunctions and aberrant tissue homeostasis was first hypothesized. Both the autosomal dominant (ADPKD) and recessive (ARPKD) variants of PKD are characterized by formation of large fluid filled cysts and greatly enlarged kidneys that are associated with increased proliferation late in the disease process. ADPKD is mainly due to mutations in the PKD1 and -2 genes, whereas ARPKD is caused by mutations in the gene encoding Fibrocystin (42). Another complex of recessive cystic kidney diseases is nephronophthisis (NPHP), resulting from defects in the genes encoding Nephrocystin (Nphp) 1-9, and being the main genetic cause of end-stage renal failure within the first three decades of life. As opposed to PKD, tubular cyst formation in NPHP does not result in enlarged kidneys, but phenotypic characteristics include degradation of tubular basement membranes, tubular collapse and interstitial fibrosis. The cystic kidney phenotype in NPHP is frequently combined with other defects such as cerebellar hypoplasia and ataxia in Joubert Syndrome, or retinitis pigmentosa in Senior-Løken Syndrome.

Many proteins whose functions are disturbed in cystic diseases have been localized to the cilium or the ciliary base body, where they might contribute to regulating kidney development and function. Included herein are the polycystins, the nephrocystins, fibrocystin, and proteins regulating Wnt signaling and planar cell polarity, which in different ways coordinate a series of signal transduction pathways in the kidney. It was proposed that PKD1 and PKD2 form a protein complex in the primary cilium to function as a mechanosensor to elicit a calcium signal in response to fluid movement through the renal tubules, where loss of cilia or mutations in the polycystins lead to cyst formation. Consistent with this, earlier studies showed that bending of primary cilia in cultures of renal epithelial duct cells by fluid shear or mechanical stimulation causes intracellular Ca2+ to increase, suggesting that ciliary mechanotransduction is important for normal function of the kidney epithelium and that loss of the cilium leads to PKD.

More recent studies in mice have revealed that the rate of cyst formation and cystic disease severity is dependent on when cilia or polycystin function is disrupted. Using conditional alleles of ift88, kif3a (component of the heterotrimeric IFT kinesin-2 motor complex), or PKD1, and inducible Cre deletor mouse lines it was shown that disruption in early postnatal life (P1 to P12) results in rapid cyst formation within three weeks of loss of the gene. In contrast, if cilia or polycystin-1 function is disrupted after P12, cyst formation requires 6 months to a year to form. There was no marked increase in proliferation rates between the mutant and control kidneys, even in the cystic animals where function was disrupted in perinatal periods. Together these data indicate that there is a critical time point at which cilia dysfunction causes a rapid or slow progressing cystogenic phenotype and raised concern about the simple pathogenic model whereby loss of cilia-mediated mechanosensation is the cause of cyst formation. In further studies, it was revealed that this switch point might be associated with completion of renal differentiation, which occurs at about two weeks of age in mice, and changes in the proliferative environment and a large change in the gene expression profile that occurs at around P12. The importance of having a proliferative environment for cyst formation in the cilia mutants was analyzed by inducing cilia loss in adult mice followed by renal injury through obstruction or ischemic reperfusion. This injury reinitiates proliferation in the adult kidney as part of the repair process and was found to result in rapid cyst formation similar to that seen in the perinatally induced cilia mutants. Cysts were not present in the contra-lateral non-injured cilia mutant kidney. A possible mechanism connecting ciliary dysfunction, cell proliferation, and cyst formation was further defined by studies of Fischer et al (Fischer E, Legue E, Doyen A, Nato F, Nicolas J F, Torres V, Yaniv M, Pontoglio M. Defective planar cell polarity in polycystic kidney disease. Nat Genet 2006; 38:21-23. 2006. [PubMed: 16341222]). They showed that mitotic spindles in the perinatal kidney normally align along the axis of the nephron such that cells divisions increase nephron length. In contrast, in cystic disease mouse and rat models the orientation of cells divisions occur randomly with many resulting in expansion of the diameter of the nephron. Deletion of the ciliary protocadherin and planar cell polarity (PCP) protein Fat4 was recently shown to elicit a similar disruption of spindle orientation during renal tubular elongation in mice. Thus, these data suggest that cilia or the basal body are needed for normal orientation of the spindle. Whether this is in response to fluid flow and the polycystin generated calcium signal, and how this is coupled to PCP has yet to be fully addressed.

Ciliopathies: Multisystem Diseases

### Joubert Syndrome

Mutations in seven genes (NPHP1, AHI1, CEP290, RPGRIP1L, MKS3/TMEM67, CC2D2A and ARL13B) and 2 additional loci (9q34, 11p12-11q13.3) have been associated with JS.

Studies have reported a genotype-phenotype correlation between MKS3 mutations and clinically apparent liver disease (elevated transaminases, portal hypertension and/or liver fibrosis on biopsy). Further, patients with NPHP1 deletions invariably have renal disease and less severe brain malformation. Most subjects with AHI1 mutations have retinal dystrophy, but very few have renal disease. In contrast, CEP290 mutations cause a spectrum of phenotypes, from isolated JS, to JS with retinal and renal disease, to severe MKS. RPGRIP11L mutations also cause a broad spectrum of disease with renal and liver involvement, but only rarely retinal dystrophy.

Mutations in RPGRIP1L, CC2D2A and MKS3/TMEM67 have been shown to cause both MKS and JS. Similarly, NPHP1 mutations can cause isolated nephronophthisis or mild forms of JS. Further, CEP290 mutations can cause JS, MKS, and BBS, as well as isolated LCA and nephronophthisis.

Many features of Joubert Syndrome (JS) (mid-hindbrain malformation, retinal dystrophy, cystic renal disease, congenital hepatic fibrosis and polydactyly) overlap with ciliopathies in general and Meckel syndrome (MKS) in particular. Studies have reported that these overlapping phenotypic features reflect a shared molecular pathophysiology involving the primary cilium and basal body (PC/BB) organelle.

Studies have reported neuropathology in JS patients. In addition to vermis hypoplasia, the most frequent structures noted to be abnormal are: the deep cerebellar nuclei, the inferior olivary nuclei, multiple cranial nerve nuclei and the decussations of the superior cerebellar peduncles and pyramidal tracts. It is not clear from the pathology which abnormalities are primary and which are secondary.

JS patients also uniformly present abnormal eye movements, though variable in severity. The presence of nystagmus, saccades instead of smooth pursuit and tracking/acquiring targets with head movements rather than eye movements are the predominant clinical signs. Quantitative eye movement recordings demonstrate that the gains (target velocity/eye velocity) for smooth pursuit, saccades, optokinetic nystagmus and vestibuloocular reflex are variably reduced. Each of these oculomotor deficits reflect abnormalities of specific neural ensembles in the cerebellar vermis (oculomotor, nodulus, uvula), flocculus/paraflocculus, deep cerebellar nuclei, vestibular nuclei, pontine nuclei and/or inferior olives. Retinal dystrophy with visual loss occurs in a subset of patients with JS.

Most reports indicate that patients with JS have substantial cognitive impairment though range of ability is quite broad.

Similar to the retinal disease seen in JS, the renal disease ranges in severity, from cystic renal dysplasia overlapping with MKS to classic nephronophthisis with onset in late childhood or later. The first sign of later onset renal disease is often failure to concentrate urine (salt-losing renal insufficiency) followed by echogenic kidneys on ultrasound and eventual renal failure. In patients with AHI1 mutations, onset of renal disease has been reported in young adults.

COACH syndrome (Cerebellar vermis hypoplasia, Oligophrenia—developmental delay/mental retardation, Ataxia, Colobomas, and Hepatic fibrosis) is considered a sub-type of JS. Clinically, the liver disease can be asymptomatic or present with mildly elevated serum transaminases, but more often, it is identified by liver imaging or signs of portal hypertension (varices, hepatosplenomegaly, ascites and rarely, upper GI bleeding). Biopsy findings are in the spectrum of the ductal plate malformation and congenital liver fibrosis, and the fibrosis is progressive at least in some cases. Severe disease requires porto-systemic shunting or liver transplantation and can result in death.

Further, polydactyly is a feature of many ciliopathies. Most frequently, it is post-axial, although it can be pre-axial or very rarely, mesaxial. In general, polydactyly is not functionally significant, and surgical correction is at the discretion of the patient/family. As in many children with hypotonia, scoliosis is a common complication and requires close monitoring, especially during puberty.

Studies have reported that the proteins encoded by all of the JS genes localize to the PC/BB (primary cilium/basal body) or directly interact with components of the PC/BB (Table 2). In addition, mutations in the mouse orthologs of two JS genes (rpgrip11 and arl13b) disrupt cilium number/morphology and cause aberrant dorsal-ventral patterning of the early neural tube, but later defects in brain development have not been explored. It remains unclear whether the human RPGRIP1L and ARL13B phenotypes are milder because the mutations identified in humans have less severe effects on overall PC/BB function, effects on a subset of PC/BB functions or effects outside the PC/BB. Disruption of cilium function by loss of ift88 function after E13 in the mouse cerebellum results in markedly decreased granule cell proliferation without disruption in overall patterning of the cerebellum or brainstem. Purkinje cells also are abnormal, potentially secondary to the granule cell defect. Conditional loss of kif3a function generates a similar phenotype, supporting a specific role for the cilium. Given the requirement for IFT proteins in granule cell proliferation, it would not be surprising if RPGRIP1L and ARL13B were also required for granule cell proliferation, potentially via effects on SHH signaling.

TABLE 2

| Gene | PC/BB function |
|---|---|
| NPHP1 | BB localization; interaction |
| | w/NPHP2-4, AHI1 |
| RPGRIP1L | PC defects; BB and PC |
| | localization; interaction w/NPHP4 |
| CEP290 | BB localization |
| TMEM67/MKS3 | PC defects; BB and PC |
| | localization; interaction with MKS1 |
| AHI1 | BB localization; interaction with |
| | NPHP1 |
| ARL13B | PC defects; PC localization |
| CC2D2A | BB localization; interaction |
| | w/CEP290, IFT88 |

BB = basal body;
PC = primary cilium.

NPHP1 encodes nephrocystin-1 which interacts with AHI1, other NPHP proteins and components of cell-cell and cell-matrix signaling pathways. Nephrocystin-1 is localized to the transition zone of the PC/BB in renal epithelia and also to the adherens junctions and focal adhesions in a cell cycle dependent manner.

RPGRIP1L encodes the RPGRIP1L protein which, like its retinally-expressed homolog RPGRIP, interacts with the NPHP4 gene product, nephrocystin-4, a ciliary protein defective in some cases of isolated nephronophthisis and Senior-Løken syndrome (nephronophthisis plus retinal dystrophy/LCA). Mutations in both RPGRIP1L and NPHP4 disrupt this interaction. RPGRIP localizes to the basal body and also colocalizes with CEP290 in brain and kidney. Knockout of the murine ortholog of RPGRIP1L, results in ciliary assembly/functional defects, abnormal left-right asymmetry and preaxial polydactyly, all potentially resulting from disturbed Shh signaling.

The protein encoded by CEP290 localizes to centrosomes and the mitotic spindle in a cell cycle dependent manner and has been shown to directly activate the ATF4 transcription factor in cultured cells. A homozygous in-frame deletion found in a murine retinal dysplasia model (rd16) abolished interaction with RPGR, a microtubule transport protein, suggesting a specific role for CEP290 in microtubule-based ciliary transport. More recently, CEP290 has been implicated in vesicle transport and cilium formation/maintenance through its interactions with PCM1105 and CP110106 as well as its role in transport of G-proteins into olfactory cilia.

TMEM67/MKS3 encodes the meckelin protein which localizes to the primary cilium and to the plasma membrane. MKS3 interacts with MKS1 and knockdown of either MKS1 or MKS3 by siRNA in ciliated renal epithelia blocked migration of the basal body to the plasma membrane and primary cilium formation. Some studies have reported that, in contrast to these results, cilia are present, albeit elongated and dysmorphic, in mks3 knockout mouse.

AHI1 encodes a protein with WD40 and SH3 domains and is highly expressed in human fetal brain and kidney. In postnatal mouse brain, expression is detected at higher levels in the cerebellar dentate nuclei and the deep cortical neurons that form the corticospinal tract, potentially correlating with the failure of decussation observed in functional and neuropathological studies of JS. AHI1 localizes to the cell junctions and centrosomes in cultured cells and interacts with NPHP1 and HAP1.

ARL13B is an ADP ribosylation factor (Arf) related gene in the Ras superfamily of small GTPases, some of which have been implicated in cytoskeletal dynamics, lipid metabolism and vesicle trafficking. ARL13B localizes to cilia in the mouse node, neural tube and fibroblasts, and an arl13b null mutation in mice causes neural tube defects, polydactyly and aberrant Shh signaling in the neural tube. Loss of function in the zebrafish scorpion mutant results in abnormal body shape and pronephric (kidney) cysts.

CC2D2A encodes a protein with similar overall structure to RPGRIP1L including coiled-coil domains, a C2 domain and an overlapping centrosomal protein related domain. CC2D2A physically interacts with CEP290 and loss of Cc2d2a function in the zebrafish sentinel mutant results in abnormal body shape and pronephric (kidney) cysts that is strongly exacerbated by knockdown of Cep290 function.

Studies have reported that BBS (Bardet-Biedel Syndrome) proteins form a complex (the BBSome) that associates with PCM1 and rab8, and is required for sorting proteins to the cilium. CEP290 has been reported to be part of the complex and is required for transporting olfactory receptors to the olfactory cilium. Other BBS proteins share homology with chaperonins and potentially are required to help build the BBSome complex. The JS gene products share protein domains with BBS gene products including coiled coils (RPGRIP1L, CC2D2A, CEP290, ARL13B, and BBS2, 4, 7, 9), WD40 repeats (AHI1 and BBS1, 2, 7) and Rab GTPase motifs (ARL13B and ARL6).

Despite numerous studies focused on various ciliopathies, many questions remain unanswered. For example, prenatal diagnosis for several ciliopathies remains an issue for many families in which the genetic cause has not been identified. Additionally, prognostic information in the literature is limited by small numbers of patients, diverse ascertainment strategies, and lack of standardized assessments. Furthermore, the neuropathological findings in humans and model organisms generally are incompletely characterized, and while certain genes are implicated in several ciliopathies, the precise molecular function of these genes remains elusive and many elements remain to be identified. The described invention addresses these issues.

### SUMMARY

According to one aspect, the described invention provides for a method for identifying a therapeutic target for treating a disease comprising a ciliopathy, the method comprising steps: (a) providing an animal model system of the ciliopathy for testing a putative therapeutic agent, wherein the animal comprises ciliated cells; (b) labeling the ciliated cells of the animal of step (a) with a traceable agent; (d) administering a disruptive agent wherein the disruptive agent affects the function of at least one therapeutic target of the animal of step (a), (e) comparing a measurable trait of the labeled ciliated cells of step (b) with a wild type animal, (f) identifying the at least one therapeutic target of step (d) as a therapeutic target for treating a ciliopathy, wherein the therapeutic target affects at least one trait of cilia or a dendrite extension of the ciliated neurons. According to one embodiment, the animal is Caenorhabditis elegans. According to another embodiment, the administering step (d) further comprises associating the disruptive agent with the at least one therapeutic target. According to another embodiment, the cell is a neuron. According to another embodiment, the measurable trait is an increase or decrease of a parameter that modulates at least one function of the therapeutic target. According to another embodiment, the measurable trait is morphology. According to another embodiment, the disruptive agent of step (d) is a double stranded RNA molecule. According to another embodiment, the method further comprises identifying a modulator of the disruptive agent of step (d), wherein the modulator at least partially affects the measurable trait of the therapeutic target. According to another embodiment, the measurable trait is morphology. According to another embodiment, the morphology is of cilia. According to another embodiment, the method further comprises administering an modulator of the disruptive agent of step (d) to a patient suffering from a ciliopathy, wherein the modulator of the disruptive agent at least partially affects the measurable trait of the therapeutic target by increasing the activity of the measurable trait. According to another embodiment, the method further comprises administering an modulator of the disruptive agent of step (d) to a patient suffering from a ciliopathy, wherein the modulator of the disruptive agent at least partially affects the measurable trait of the therapeutic target by decreasing the activity of the measurable trait. According to another embodiment, the traceable agent is a lipophilic dye. According to another embodiment, the traceable agent is According to another embodiment, the traceable agent is a fluorescence protein. According to another embodiment, the ciliopathy is a cil-1-dependent ciliopathy. According to another embodiment, the ciliopathy is the Joubert Syndrome. According to another embodiment, the ciliopathy is congenital hepatic fibrosis/Caroli Syndrome. According to another embodiment, the ciliopathy is an autosomal dominant polycystic kidney disease. According to another embodiment, the ciliopathy is nephronophthisis. According to another embodiment, the ciliopathy is Bardet-Biedhl Syndrome.

### BRIEF DESCRIPTION OF THE DRAWINGS

FIG. 1 shows the ciliated neurons in C. elegans.

FIG. 2 shows movement of ciliary components along the axoneme.

FIG. 3 shows ciliated neurons in anterior part (A) (ASK, AD, ASI, AWB, ASJ) and posterior part (B) (PHA, PHB) of C. elegans labeled with a lipophilic fluorescent dye.

FIG. 4 shows three groups of genes with Transition Zone (TZ) localizing products. These groups were categorized based on phasmid dye filling data.

FIG. 5 shows dissimilar ciliary phenotypes (synthetic dye filling phenotype, SynDyf) in amphids (head) and phasmids (tail).

FIG. 6 shows synthetic dye filling phenotypes (phasmids and amphid) of mks-6 and double mutants with other mks genes.

FIG. 7 shows genetic interaction of cil-1 with nphp-2 based upon synthetic dye filling phenotype (SynDyf).

FIG. 8. shows that cil-1 is required for TRP polycystin complex localization. (A and B) Cartoons illustrating locations and structure of pkd-2-expressing neurons in C. elegans male head (A) and tail (B). (C and D) In a wild type male, PKD-2::GFP localizes to cilia and neuronal cell bodies of CEM, ray B (RnB), and hook B (HOB) neurons. (E and F) In cil-1(my15) males, PKD-2::GFP is abnormally distributed along neurons including dendrites and axons. PKD-2::GFP in ciliary regions appears to be wild type. (G) In lov-1 CEMs, PKD-2::GFP accumulates in cell bodies and weakly labels in cilia. (H) In lov-1 RnBs, PKD-2::GFP aggregates in cell bodies and distributes along dendrites and cilia. (I) In cil-1; lov-1 CEMs, PKD-2::GFP aggregates in cell bodies and distributes along dendrites and cilia. (J) In cil-1; lov-1 RnBs, PKD-2::GFP forms bright aggregates in the cell bodies, similar to the lov-1 single mutant. (K) In stam-1 CEMs, PKD-2::GFP accumulates in ciliary regions. (L) In stam-1 RnBs, PKD-2::GFP accumulates in the ciliary regions and distal dendrites. (M) In stam-1; cil-1 CEMs, PKD-2::GFP localizes to dendrites and axons and sometimes accumulates ciliary bases. (N) In stam-1; cil-1 RnBs, PKD-2::GFP is distributed to dendritic and axonal processes, similar to cil-1 single mutants.

FIG. 9 shows localization of LOV-1::GFP and Ppkd::SNB-1::GFP, and mating behavioral efficiency of mutants. (A, B) In WT, LOV-1::GFP is found in cell bodies of CEM and RnB neurons and cilia of CEM neurons. Only one CEM cilium is brightly labeled with LOV-1::GFP, probably due to low LOV-1::GFP expression levels or mosaicism. (C) In cil-1(my15) CEMs, LOV-1::GFP is visible. in dendrites and axons as well as cell bodies. (D) In cil-1(my15) RnBs, LOV-1::GFP is found in dendrites and cell bodies. (E, F) Ppkd-2::SNB-1(Synaptobrevin)::GFP in CEM neurons of both WT and cil-1(my15) animals localizes to the axonal processes. (G) Mating behavioral efficiency of WT (him-5(e1490)), pkd-2(sy606); him-5(e1490), and cil-1(my15); him-5(e1490). cil-1; him-5 males exhibit WT behaviors in three steps during mating behaviors (response, vulva location, and spicule insertion/initiation of sperm transfer). Response and vulva location steps require pkd-2 and lov-1. Error bars for response and vulva location efficiencies reflect the standard errors between independent experimental groups, each consisting of at least 20 animals.

FIG. 10 shows genetic and physical maps of cil-1 locus. (A) Genetic and physical maps of the region of LG III encompassing the cil-1 locus. Positions of rescuing genomic fragments are aligned approximately to the physical map. (B) Summary table for rescue effects of injected cil-1(my15) lines. The Spe phenotype was scored by hermaphroditic brood size. (C) C50C3.7 is the gene mutated in cil-1(my15). Six blank boxes indicate exons connected with five introns. my15 is a nonsense mutation in the fifth exon (301st amino acid). The black box at the end of the third exon indicates the position of alternative splicing for the short form C50C3.7b. C50C3.7a encodes a phosphoinositide 5-phosphatase.

FIG. 11 shows phylogenetic analysis on 5-phosphatases. (A) Phylogenetic analysis of 5-phosphatase family. The NJ tree based on the analysis of the catalytic domain of inositol 5-phosphatase protein sequences encoded in the genomes of five animals and three related unicellular eukaryotes. Type I sequences were excluded in the analysis due to their low sequence similarity to the sequences of interest. Although the catalytic domain sequences of the RhoGAP domain-containing inositol 5-phosphatases of C. elegans and D. melanogaster did not form a Glade with the other Type II/OCRL sequences, they nevertheless share several amino acids within a hypervariable region, suggesting they are derived from a common ancestor. Catalytic domains are highly homologous between CIL-1 and T25B9.10, but the latter lacks the SKICH-like domain. The tree is arbitrarily rooted. Bootstrap support values (NJ/MP) over 50% are shown at the corresponding nodes. Note that the presence of characteristic domains is indicated with symbols as designated in rectangular boxes. (B) Two conserved sequence motifs of 5-phosphatases. Representative members of each subfamily and SKICH-like member are aligned. Pink vertical boxes indicate residues with absolute conservation including CIL-1. Four residues with arrows are essential residues for 5-phosphatase catalytic activity. The residue in red (N) was substituted to A to create the phosphatase-DEAD CIL-1 construct. (C) Predicted phylogenetic relationships among eight organisms included in the study. Note that relationships among nematodes (e.g., C. elegans), arthropods (e.g., D. melanogaster), and deuterostomes (e.g., vertebrates) remain controversial (dotted line). (D) SKICH-like domain alignment. Pink vertical boxes indicate identified residues in mammalian PIPP and SKIP and gray boxes include residues with reported similarities between mammalian members. Open boxes indicate additional homologous sequences. The CIL-1 SKICH-like domain is slightly divergent.

FIG. 12 shows cil-1 expression and localization. (A-C) The GFP expression driven by a 2.2 kb bath-42 promoter (Pbath-42(2.2 kb)::GFP) is broad in both hermaphrodites and males. In adult hermaphrodite head (A), adult male head (B), and tail (C), the 2.2 kb bath-42 promoter is expressed in the pharynx (ph), intestine (in), and unidentified neurons (n). (D-E) Ppkd-2::CIL-1::tdTomato in male head (D) and tail (E). The tdtomato labels cilia (arrowheads), dendrites (den), axon, and small spots in cell bodies (arrows). (F) Intestinal expression of CIL-1 labels reticular structure in the cytoplasm (arrows). Scale bar, 10 um.

FIG. 13 shows that cil-1 regulates PI(3,5)P2 and PI(3,4,5)P3 subcellular distribution. GFP-tagged PI-specific markers in the intestine of adult males are 2×FYVE for PI(3)P, AKT (PH domain) for PI(3,4,5)P3 and PI(3,4)P2, and PLCdelta (PH domain) for PI(4,5)P2. (A-C) In the WT intestine: (A) PI(3)P labels meshlike, tubulovesicular structures in the cytoplasm without obvious PM labeling. (B) PI(3,4,5)P3 and PI(3,4)P2 label similar tubulovesicular structures as well as the apical (arrowheads) and basolateral (arrow) PM. (C) PI(4,5)P2 predominantly labels the apical (arrowheads) in addition to the basolateral (arrow) PM. (D-F) In the cil-1(my15) intestine: (D) PI(3)P appears soluble in the cytoplasm. (E) PI(3,4,5)P3 and PI(3,4)P2 lose their tubulovesicular pattern, appearing diffuse in the cytoplasm. The PM labeling is less prominent in cil-1 mutants (arrowheads). (F) PI(4,5)P2 remains enriched in the PM. The scale bar represents 10 mm. (G and H) tdTomato-tagged 2×FYVE domain [PI(3)P marker] expression in male-specific neurons of WT and cil-1(my15). (G) In WT CEMs, the PI(3)P marker is bright in the nuclei (denoted as “N”), labels small puncta in the cell bodies, but is almost absent from cilia (blank yellow arrowhead). (G0) Similarly, in WT RnBs, the PI(3)P marker is confined to cell bodies (nuclei and small puncta). (H) In cil-1(my15) CEMs, the PI(3)P marker is visible in cilia and dendrites (arrows) in addition to cell bodies. The inset shows PI(3)P marker labeling ciliary and dendritic regions. (HO) In cil-1(my15) RnBs, the PI(3)P marker is occasionally visible in cilia (yellow arrowhead) in addition to cell bodies

FIG. 14 shows that PI and early endosomal markers in male specific sensory neurons. Arrows indicate the location of cell bodies. (A-B) Ppkd-2::AKT(PH)::GFP as a PI(3,4,5)P3/PI(3,4)P3 marker. male tail. (A) PI(3,4,5)P3/PI(3,4)P3 in WT male tail neurons is primarily visible on cell surface. (B) In cil-1(my15), PI(3,4,5)P3/PI(3,4)P3 is similarly distributed along the plasma membrane in male tail neurons. (C-D) Ppkd-2::PLCδ(PH)::GFP labels PI(4,5)P2 in male tail neurons. In both WT (C) and my15 (D) neurons, PI(4,5)P2 is evenly distributed in the cell surface. (E-F) Ppkd-2::FYVEx2::GFP as a PI(3)P marker. (E) In WT, PI(3)P is bright in the nuclei (arrows, big spots) and small compartments supposedly early endosomes, but rarely visible in dendritic processes or cilia. (F) In my15, PI(3)P is similarly bright in the nuclei (arrows), but distinctly labels distal dendrites and cilia (arrowheads). (G-H) Ppkd-2::mRFP::RAB-5 labels early endosomes (arrows, small spots) and smoothly along the dendrites (den) both in WT (G) and my15 (H) male tail neurons. (J-I) Similarly, Ppkd-2::STAM-1::mRFP is visible in early endosomes (arrows) and dendrites (den) in WT (J) and my15 (I) background.

FIG. 15 shows that cil-1(my15) mutants are semi sterile due to sperm defects. (A) Semi-sterility in cil-1(my15); him-5(e1490). my15 hermaphrodites have a reduced brood size at 20 degree compared to WT. (B) my15 hermaphroditic sterility can be rescued by mating with WT males. (C) my15 males exhibit a low Mating Efficiency (% of cross progeny over total progeny), measuring males' ability to sire cross progeny. When mated with my15 males, the number of total progeny is not suppressed, indicating that sperm from my15 males do not compete with endogenous hermaphrodite-derived sperm. (D-E) DAPI stained adult hermaphrodites. (D) In a WT hermaphrodite, the gonad contains developing embryos (arrowheads). Spt; location of the spermatheca. (E) my15 hermaphrodites contain endomitotic oocytes (arrowheads) in the uterus, showing large DAPI positive spots. Progression of germline development appears normal both in my15 hermaphrodites and male; however, the morphology of germline tube may be mildly affected, suggesting pleiotropic roles of cil-1. Panel D and E are images of young adult hermaphrodites under the same magnification. In E, however, the my15 unfertilized oocytes are prominent (arrowheads) and the my15 gonad appears slightly narrower than WT, possibly due to massive unfertilized oocyte accumulation or unknown cil-1 function in maintaining healthy gonad architecture. Scale bar, 50 um. (F-G) Nomarski (differential interference contrast (DIC)) images of WT and my15 hermaphrodite uterus. Spermatheca (not shown) to the left, vulva to the right as indicated. (F) The WT hermaphrodite uterus contains a zygote, 2-cell and 4-cell embryo, which are encapsulated with eggshell (arrowheads). (G) In the my15 hermaphrodite uterus, endomitotic cells are identified by large nuclei (arrowheads), lack of eggshell, and irregular shapes. Scale bar, 10 um.

FIG. 16 that cil-1 positively regulates sperm activation and motility. (A) C. elegans sperm activation summarized in this illustration depicting pathways and genes functioning during sperm activation and fertilization. In a spermatid, MOs containing a TRPC receptor, TRP-3, are located just below the PM. During WT activation (solid arrow), MOs fuse to the PM and a pseudopod develops, producing a motile spermatozoon. In fer-1 mutant sperm (upper dotted arrow), MOs do not fuse with the PM and a short pseudopod forms, resulting in immotile sperm. A cil-1 mutant sperm (lower dotted arrow) is normal in MO fusion but develops into immotile spermatozoon with a short pseudopod. TRPC TRP-3 translocation from MO to the PM appears normal in cil-1 mutant sperm. spe-9, spe-38, spe-41/trp-3, and spe-42 encode various membrane proteins required for sperm-egg interactions. Loss of any of these genes results in motile but infertile spermatozoa. (B and C) Nomarski images of isolated male-derived sperm before and after in vitro activation and endogenously activated hermaphrodite-derived sperm. (B) shows a round WT spermatid. (B′) shows WT spermatozoa after 15 min of pronase activation. Spermatozoa extend full-length pseudopods (yellow arrowheads). Yellow bars depict the length of WT spermatozoa measured. (B″) shows WT hermaphrodite-derived sperm that are endogenously activated. (B″′) WT male-derived sperm are activated to spermatozoa with pseudopods (arrowarrowheads) within 10 min of 100 nM wortmannin application. (C) cil-1(my15) mutant spermatids before activation are slightly smaller than WT spermatids. (C′) Upon activation, cil-1 mutant sperm develop stubby pseudopods. The length of sperm is indicated with orange bars shorter than WT (compare to yellow bars). (C″) Hermaphrodite-derived sperm from the cil-1(my15) mutant occasionally develop short pseudopods. (C″′) my15 male-derived sperm are not activated by 100 nM wortmannin but exhibit subtle morphological changes. (D) Sperm-tracking assay. The majority of WT male-derived sperm are deposited and retained within the spermatheca in the hermaphroditic reproductive tract at 10 and 16 hr. In contrast, cil-1(my15) male-derived sperm are not found in the spermatheca at 16 hr. spt denotes spermatheca. (E and E′) Ultrastructure of him-5(e1490) (E) and cil-1(my15) him-5(e1490) (E′) spontaneously activated spermatozoa. The following abbreviations are used: lm, laminar membranes; mo, membranous organelles; n, nucleus; and p, pseudopod. Although the cytoplasm in this cil-1 pseudopod (compare E to E′) appears denser than that of the WT control, the significance, if any, of this observation is unclear. (F and G) Monitoring MO fusion during sperm activation with a lipophilic FM1-43 dye. (F) The PM of WT spermatids is stained with FM1-43. (F0) In WT spermatozoa, the dye concentrates at the MO fusion sites. MO fusion events are restricted to the PM of cell body (arrowheads) but not pseudopod (bracket). (G) FM1-43 dye marks the PM of cil-1 spermatids. (G′) In short my15 spermatozoa with visible pseudopods, MO fusion sites are concentrated on the cell body (arrowheads) and excluded from the pseudopod PM (bracket). (H and I) Immunohistochemistry of sperm with the MO antibody 1CB4 (red), anti-TRP-3 (green), and DAPI (blue). The triple-labeled images were generated by overlaying three confocal images from the same Z section. (H) In WT spermatids, MOs (red) are located around the cell periphery below the PM. The location of TRP-3 (green) partially overlaps with 1CB4-labeled MOs. (H′) In WT spermatozoa, 1CB4-positive MOs are primarily located around the PM but absent from pseudopods (compare with bracket area in H″). (H″) In WT spermatozoa, anti-TRP-3 staining is detectable in the cell body and pseudopod PM(bracket). (H″′) An overlay of WT spermatozoa is shown. (I) In my15 spermtids, MOs (red) are localized to the cell periphery just below the PM as in WT. Anti-TRP-3 staining (green) overlaps with MOs. (I′) In my15 spermatozoa, similar to WT, MOs are found in the cell body but not pseudopod. (I″) In my15 spermatozoa, TRP-3 protein is detected both in the cell body and in the pseudopod, as in WT. (I′) An overlay of (H′) and (H″) with the DAPI image is shown. The scale bars represents 5 mm.

FIG. 17 shows models of CIL-1 functions (A and B) A model for CIL-1 function in polycystin trafficking in sensory neurons. (A) In WT RnB neurons, polycystins are (1) assembled in the cell body, transported along the dendrite, and (2) exocytosed at the PM, presumably at the ciliary base. Ciliary abundance of polycystins is tightly regulated by dynamic (3) internalization and endocytosis. (4) Pre-early endosomal (pre-EE) vesicles are sorted to PI(3)P-enriched (red lining) EE, followed by (5) targeting to multivesicular body (MVB). CIL-1 functions to maintain the balance between PI(3)P and PI(3,5)P2 in the EE and MVBmembrane. Polycystin sorting from EE to MVB requires the STAM/Hrs complex. (6) Lysosomal degradation downregulates the polycystins. In CEM neurons, cil-1 act at least partially in parallel with lov-1 and stam-1, as indicated with green. (B) In cil-1(my15), the Cil defect may occur after endocytosis. Loss of CIL-1 function causes depletion of PI(3)P, which in turn affects EE biogenesis and maturation. Pre-EE vesicles form an excessively tubularized EE along the microtubule network. Alternatively, polycystin-containing pre-EE vesicles may accumulate in neurons because their destination point [PI(3)P-enriched EE] is blocked. In my15 CEM neurons, loss of cil-1 affects dendritic targeting and postendosomal degradation (in green). (C and D) A Model for CIL-1 function in sperm activation. (C) In a WT spermatozoon, lowering PI(3,4,5)P3 (yellow) by CIL-1 action initiates unidentified signaling pathway(s) to coordinate pseudopod extension and sperm movement. An unidentified 3-kinase that generates PI(3,4,5)P3 negatively regulates sperm activation. The balanced action between CIL-1 and 3-kinase maintains low levels of PI(3,4,5)P3, permitting sperm activation. (D) In cil-1 mutant sperm, abnormally high PI(3,4,5)P3 levels inhibit downstream signaling pathways for pseudopod extension and sperm motility.

### DETAILED DESCRIPTION

The described invention provides an animal model system and methods of utilizing Caenorhabditis elegans cil-1 and the corresponding protein CIL-1, for identifying therapeutic targets for treating a ciliopathy in a mammal. The Caenorhabditis elegans cil-1 gene encodes CIL-1, a phosphoinositide 5-phosphatase. This enzyme regulates phosphoinosides, which are important in many cellular processes and human diseases. In C. elegans, cil-1 is required for ciliary trafficking of the TRP polycystins (in humans, these genes are mutated in autosomal dominant polycystic kidney disease) and sperm activation. The C. elegans cil-1-based animal system described herein provides a model system for human ciliopathies, including those of INPP5E, which when mutated in humans results in Joubert's Syndrome.

### Glossary

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Glutamic acid; G=Gly=Glycine; H=His=Histidine; I=Ile=lsoleucine; L=Leu=Leucine; K=Lys=Lysine; M=Met=Methionine; F=Phe=Phenyalanine; P=Pro=Proline; S=Ser=Serine; T=Thr=Threonine; W=Trp=Tryptophan; Y=Tyr=Tyrosine; V=Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide.

The following represent groups of amino acids that are conservative substitutions for one another:

Alanine (A), Serine (S), Threonine (T);

Aspartic Acid (D), Glutamic Acid (E);

Asparagine (N), Glutamic Acid (Q);

Arginine (R), Lysine (K);

Isoleucine (I), Leucine (L), Methionine (M), Valine (V); and

Phenylalanine (F), Tyrosine (Y), Tryptophan (W).

The terms “administering” or “administration” as used herein are used interchangeably to mean the giving or applying of a substance and include in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing the conventional nontoxic pharmaceutically acceptable carriers, adjuvants, and vehicles as desired, or may be locally administered by means such as, but not limited to, injection, implantation, grafting, topical application, or parenterally. The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

Additional administration may be performed, for example, intravenously, pericardially, orally, via implant, transmucosally, transdermally, intramuscularly, subcutaneously, intraperitoneally, intrathecally, intralymphatically, intralesionally, or epidurally. Administering can be performed, for example, once, a plurality of times, and/or over one or more extended periods.

The term “associate” or any of its grammatical forms as used herein refers to joining, connecting, or combining to, either directly, indirectly, actively, inactively, inertly, non-inertly, completely or incompletely.

The term “attenuate” as used herein refers to render less virulent, to weaken or reduce in force, intensity, effect or quantity.

The terms “carrier” and “pharmaceutical carrier” as used herein refer to a pharmaceutically acceptable inert agent or vehicle for delivering one or more active agents to a mammal, and often is referred to as “excipient.”

The term “cell” is used herein to refer to the structural and functional unit of living organisms and is the smallest unit of an organism classified as living.

The term “ciliopathy” as used herein refers to a genetic disorder of cellular cilia or basal bodies.

The term “cil-1-dependent ciliopathy” as used herein refers to a ciliopathy that is a result, a disease, a disorder, a symptom, a syndrome, a pathology, or other abnormality caused or influenced by a mutation or abnormal function of the cil-1 gene, or variant(s), homolog(s), and ortholog(s) thereof. For example, cil-1-dependent ciliopathies include, but are not limited to, Joubert Syndrome, a liver disease, congenital hepatic fibrosis/Caroli Syndrome, polycystic liver disease, a kidney disease, autosomal dominant polycystic kidney disease (ADPKD) autosomal recessive polycystic kidney disease (ARPKD), nephronophthisis (NPHP), Bardet-Biedhl Syndrome (BBS), glomerulocystic kidney disease (GCKD), cystic dysplastic kidneys (CDK), multicystic dysplastic kidneys (MCDK), and medullary sponge kidney (MSK).

The term “compare” as used herein refers to the examination of two or more objects, substances, molecules, states, proteins, nucleic acids, peptides, antibodies, segments, or subjects in order to note similarities and/or differences.

The term “condition” as used herein refers to a variety of health states and is meant to include disorders or diseases caused by any underlying mechanism or disorder, injury, and the promotion of healthy tissues and organs.

The term “conserved segment” is used herein to refer to similar or identical sequences that may occur within nucleic acids, proteins or polymeric carbohydrates within multiple species of organism or within different molecules produced by the same organism.

The term “contact” as used herein refers to a state or condition of touching or of being in immediate or local proximity. The term “contacting” as used herein refers to bringing or putting in contact, or to being in or coming into contact. Contacting a composition to a target destination, such as, but not limited to, an organ, tissue, cell, or tumor, may occur by any means of administration known to the skilled artisan.

The term “controlled release” is intended to refer to any drug-containing formulation in which the manner and profile of drug release from the formulation are regulated.

The term “controllable regulatory element” as used herein refers to nucleic acid sequences capable of effecting the expression of the nucleic acids, or the peptide or protein product thereof. Controllable regulatory elements may be operably linked to the nucleic acids, peptides, or proteins of the present invention. The controllable regulatory elements, such as, but not limited to, control sequences, need not be contiguous with the nucleic acids, peptides, or proteins whose expression they control as long as they function to direct the expression thereof. Thus, for example, intervening untranslated yet transcribed sequences may be present between a promoter sequence and a nucleic acid of the present invention and the promoter sequence may still be considered “operably linked” to the coding sequence. Other such control sequences include, but are not limited to, polyadenylation signals, termination signals, and ribosome binding sites.

The terms “cultivate” as used herein refers to promote or improve the growth of, or to produce by culture.

The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.” For example, delayed absorption of a parenterally administered drug form may be accomplished by dissolving or suspending the drug in an oil vehicle.

The term “disease” or “disorder” are used interchangeably herein to refer to an impairment of health or a condition of abnormal functions. The term “diseased state” as used herein refers to being in a condition of disease or disorder. The term “syndrome” as used herein refers to a pattern of symptoms indicative of some disease or condition. The term “condition” as used herein refers to a variety of health states and is meant to include disorders or disease caused by any underlying mechanism or disorder.

The term “disposed” as used herein means to place, set or arrange in a particular order.

The term “drug” as used herein refers to a therapeutic agent or any substance, other than food, used in the prevention, diagnosis, alleviation, treatment, or cure of disease. A drug is: (a) any article recognized in the official United States Pharmacopeia, official Homeopathic Pharmacopeia of the United States, or official National Formulary, or any supplement to any of them; (b) articles intended for use in the diagnosis, cure, mitigation, treatment, or prevention of disease in man or other animals; (c) articles (other than food) intended to affect the structure or any function of the body of man or other animals, and d) articles intended for use as a component of any articles specified in (a), (b) or (c) above.

The term “efficacy” as used herein refers to effectiveness, and means a therapeutic agent is therapeutically effective. Generally, a greater level of efficacy will be achieved by increasing the dose and/or frequency of administration of a therapeutic agent given to a population, such that a greater proportion of the population will receive a benefit and/or there will be a greater magnitude of benefit in an individual patient, or cell. If a first therapeutic agent is more potent than a second therapeutic agent, it will reach a greater level of efficacy than the second therapeutic agent using identical amounts of each.

The terms “expose” as used herein refers to subjecting or allowing to be subjected to an action, influence, or condition.

The term “function” as used herein refers to the activity or action of. For example, biological, chemical or physical action or activity.

The term “gene” as used herein refers to a segment of DNA that is involved in producing a polypeptide chain; it can include regions preceding and following the coding DNA as well as introns between the exons.

The terms “gene expression” and “expression” are used interchangeably herein to refer to the process by which inheritable information from a gene, such as a DNA sequence, is made into a functional gene product, such as protein or RNA.

The term “growth” as used herein refers to a process of becoming larger, longer or more numerous, or an increase in size, number, or volume of cells in a cell population.

The term “hybridization” refers to the binding of two single stranded nucleic acid molecules to each other through base pairing. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable. The effects of base incompatibility may be measured by quantifying the rate at which two strands anneal; this may provide information as to the similarity in base sequence between the two strands being annealed.

The terms “inhibiting”, “inhibit” or “inhibition” as used herein are used to refer to reducing the amount or rate of a process, to stopping the process entirely, or to decreasing, limiting, or blocking the action or function thereof. Inhibition may include a reduction or decrease of the amount, rate, action function, or process by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99% when compared to a reference substance, wherein the reference substance is a substance that is not inhibited.

The term “identifying” as used herein refers to designating, recognizing, or attributing.

The term “isolated” refers to a material, such as a nucleic acid, a peptide, or a protein, which is: (1) substantially or essentially free from components that normally accompany or interact with it as found in its naturally occurring environment. The terms “substantially or essentially free” are used to refer to a material, which is at least 80% free from components that normally accompany or interact with it as found in its naturally occurring environment. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state. For example, a naturally occurring nucleic acid becomes an isolated nucleic acid if it is altered, or if it is transcribed from DNA that has been altered, by means of human intervention performed within the cell from which it originates. See, for example, Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (for example, a promoter) becomes isolated if it is introduced by non-naturally occurring means to a locus of the genome not native to that nucleic acid. Nucleic acids that are “isolated” as defined herein are inclusive of those termed “heterologous” nucleic acids.

The term “labeling” as used herein means marking, characterizing, classifying, identifying or designating.

The term “long-term” release, as used herein, means that an implant is constructed and arranged to delivery of therapeutic levels of an active ingredient occur for at least 7 days, or for about 30 to about 60 days.

The term “mammalian cell” as used herein refers to a cell derived from an animal of the class Mammalia. As used herein, mammalian cells may include normal, abnormal and transformed cells. Examples of mammalian cells utilized within the present invention, include, but are not limited to, neurons, epithelial cells, muscle cells, blood cells, immune cells, stem cells, osteocytes, endothelial cells and blast cells. Cells may be utilized in vivo or in vitro.

The term “measure” as used herein refers to any standard of comparison, estimation or judgement, or to ascertain the extent, dimensions, quantity, or capacity by comparison with a standard. The term “measurable” means an identifiable property of an object, set, or event that is subject to being measured.

The term “minimizing progression” as used herein refers to reducing the amount, extent, size, or degree of development of a sequence or series of events.

The term “modulate” as used herein means to regulate, alter, adapt, or adjust to a certain measure or proportion.

The term “nucleic acid” as used herein refers to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” as used herein refers to a chemical compound that consists of a heterocyclic base, a sugar, and one or more phosphate groups. In the most common nucleotides the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. The purines include adenine (A), and guanine (G); the pyrimidines include cytosine (C), thymine (T), and uracil (U).

The term “operably linked” as used herein refers to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, are contiguous and in the same reading frame.

The term “overexpressed” as used herein refers to increased quantity of a gene or gene product.

The term “peptide” as used herein refers to a polymer formed from the linking together, in a defined order, of amino acids. The link between one amino acid residue and the next is known as an amide or peptide bond. By some conventions, for example, a peptide is a short polymer, of at least 2 amino acids, a “polypeptide” is a single chain of amino acids, and a “protein” contains one or more polypeptides. The term peptide as used herein is inclusive of a polypeptide, a protein or a peptidomimetic. These terms apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein that protein is specifically reactive to antibodies elicited to the same protein but consisting entirely of naturally occurring amino acids. The terms polypeptide, peptide and protein also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

The term “peptide” is used herein to refer to two or more amino acids joined by a peptide bond.

The term “polypeptide” is used herein to refer to a peptide containing about 10 to more than about 100 amino acids.

The term “protein” is used herein to refer to a large complex molecule or polypeptide composed of amino acids. The sequence of the amino acids in the protein is determined by the sequence of the bases in the nucleic acid sequence that encodes it.

The term “peptidomimetic” as used herein refers to a small protein-like chain designed to mimic a peptide. A peptidomimetic typically arises from modification of an existing peptide in order to alter the molecule's properties.

The term “pharmaceutical composition” as used herein refers to a composition that is employed to prevent, reduce in intensity, cure or otherwise treat a target condition, syndrome, disorder or disease.

The term “pharmaceutically acceptable carrier” as used herein refers to one or more compatible solid or liquid filler, diluents or encapsulating substances which are suitable for administration to a human or other vertebrate animal.

The term “pharmaceutically acceptable salt” as used herein refers to those salts which are, within the scope of sound medical judgment, suitable for use in contact with the tissues of humans and lower animals without undue toxicity, irritation, allergic response and the like and are commensurate with a reasonable benefit/risk ratio.

The term “polynucleotide” refers to a deoxyribopolynucleotide, ribopolynucleotide, or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide may be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The term “prevent” as used herein refers to effectual stoppage of action or progress.

The term “reduce” or “reducing” as used herein refers to a decrease in size, to a slowing of the growth or proliferative rate, and to a lowering in degree, or intensity.

The term “regulatory sequence” (also referred to as a “regulatory region” or “regulatory element”) refers to a promoter, enhancer or other segment of DNA where regulatory proteins, such as transcription factors, bind preferentially to control gene expression and thus protein expression.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity”.

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Clayerie and States, Comput. Chem., 17:191-201 (1993)) low-complexity filters may be employed alone or in combination.

As used herein, “sequence identity” or “identity” in the context of two nucleic acid or polypeptide sequences refers to the residues in the two sequences which are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) relative to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid.

The terms “substantial identity” in the context of a peptide indicates that a peptide comprises a sequence with at least 70% sequence identity to a reference sequence, at least 80%, at least 85%, at least 90% or 95% sequence identity to the reference sequence over a specified comparison window. Optionally, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions that are not identical may differ by conservative amino acid changes.

The term “shRNA” as used herein means a small hairpin RNA or short hairpin RNA.

“Antisense RNA” is a single-stranded RNA that is complementary to a messenger RNA (mRNA) strand transcribed within a cell.

The term “Morpholino” as used herein refers to an antisense oligomer that blocks gene expression by interfering with the translation initiation complex or with RNA splicing.

The term “mutagenic agent” as used herein means any chemical substance or physical agent that is capable of enhancing the frequency of detectable mutants within a population of organisms or cells.

The term “similar” is used interchangeably with the terms analogous, comparable, or resembling, meaning having traits or characteristics in common.

The term “stimulate” as used herein refers to activate, provoke, or spur. The term “stimulating agent” as used herein refers to a substance that exerts some force or effect.

The term “traceable agent” as used herein means a lipophilic dye, a fluorescence protein, a green fluorescent protein, or other such compound, protein, or the like that allows identification, or labeling of a compound, molecule, protein, or and the like.

The terms “residue” or “amino acid residue” or “amino acid” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

The terms “variants”, “mutants”, and “derivatives” are used herein to refer to nucleotide sequences with substantial identity to a reference nucleotide sequence. The differences in the sequences may by the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce polypeptide variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan. As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “substitution” is used herein to refer to that in which a base or bases are exchanged for another base or bases in the DNA. Substitutions may be synonymous substitutions or nonsynonymous substitutions. As used herein, “synonymous substitutions” refer to substitutions of one base for another in an exon of a gene coding for a protein, such that the amino acid sequence produced is not modified. The term “nonsynonymous substitutions” as used herein refer to substitutions of one base for another in an exon of a gene coding for a protein, such that the amino acid sequence produced is modified.

The terms “deletion” and “deletion mutation” are used interchangeably herein to refer to that in which a base or bases are lost from the DNA.

The term “addition” as used herein refers to the insertion of one or more bases, or of one or more amino acids, into a sequence.

The term “solvate” as used herein refers to a complex formed by the attachment of solvent molecules to that of a solute.

A “solution” generally is considered as a homogeneous mixture of two or more substances. It is frequently, though not necessarily, a liquid. In a solution, the molecules of the solute (or dissolved substance) are uniformly distributed among those of the solvent.

The term “solvent” refers to a substance capable of dissolving another substance (termed a “solute”) to form a uniformly dispersed mixture (solution).

The term “specifically hybridizes” as used herein refers to the process whereby a nucleic acid distinctively or definitively forms base pairs with complementary regions of at least one strand of DNA that was not originally paired to the nucleic acid. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, at least 85% sequence identity, at least 90% sequence identity, at least 95% sequence identity, or at least 100% sequence identity (i.e., complementary) with each other.

The term “subject” or “individual” or “patient” are used interchangeably to refer to a member of an animal species of mammalian origin, including but not limited to, a mouse, a rat, a cat, a goat, sheep, horse, hamster, ferret, pig, a dog, a guinea pig, a platypus, a rabbit and a primate, such as, for example, a monkey, ape, or human.

The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period.

The term “substantially” as used herein refers to considerably or significantly, within a defined range. For example, such a range can include, but is not limited to, within 10% as compared to a control or reference value, within 20% as compared to a control or reference value, within 30% as compared to a control or reference value, within 40% as compared to a control or reference value, within 50% as compared to a control or reference value, within 60% as compared to a control or reference value, within 70% as compared to a control or reference value, within 80% as compared to a control or reference value, within 90% as compared to a control or reference value, or within 99% as compared to a control or reference value.

The term “syndrome” as used herein refers to a pattern of symptoms indicative of some disease or condition.

The term “therapeutic agent” as used herein refers to a drug, molecule, nucleic acid, protein, composition or other substance that provides a therapeutic effect.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50 which describes the dose in a particular dosage that is therapeutically effective for a particular disease manifestation in 50% of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction or elimination of the progression of a disease manifestation.

The terms “therapeutically effective amount” and “pharmaceutically effective amount” are used interchangeably to refer to the amount that results in a therapeutic beneficial effect. The term as used herein shall also mean the dosage of a therapeutic agent which directly or indirectly reduces or increases the activity of molecules secreted by diseased and/or non-diseased cells participating in a disease manifestation, such that the amount of therapeutic agent arrests, reduces, or eliminates altogether the degree of the disease manifestation. Typically, a therapeutically effective amount will also eliminate, reduce, or prevent the progression of one or more diseases. A skilled artisan recognizes that in many cases a therapeutic agent may not provide a cure, but may only provide a partial benefit. Furthermore, the skilled artisan recognizes that because individual patients and disease states may vary, some patients may receive little, or no benefit at all. A dosage of therapeutic agent that “kills,” “arrests,” “reduces,” or “eliminates” as described above, in at least some patients, is considered therapeutically effective. The term “dosage” as used herein refers to the dose or amount, and frequency of administering of a therapeutic agent in prescribed amounts. The term “dose” as used herein refers to the amount of therapeutic agent to be taken or applied all at one time or in fractional amounts within a given period.

The term “therapeutic target” as used herein refers to a native protein, molecule, compound, nucleic acid, ligand, receptor, organelle, or cell whose activity optionally affect the function of the cilia of a cell, and is modified by an agent (meaning a disrupting agent, a therapeutic agent, or agent with a biological activity) resulting in a desirable therapeutic effect.

The terms “topical administration” and “topically applying” as used herein are used interchangeably to refer to delivering a peptide, a nucleic acid, or a vector comprising the peptide or the nucleic acid onto one or more surfaces of a tissue or cell, including epithelial surfaces. Although topical administration, in contrast to transdermal administration, generally provides a local rather than a systemic effect, the terms “topical administration” and “transdermal administration” as used herein, unless otherwise stated or implied, are used interchangeably.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “isolate” as used herein refers to a process of obtaining a substance, molecule, protein, peptide, nucleic acid, or antibody that is substantially pure and is free of other substances with which it is ordinarily found in nature or in vivo systems to an extent practical and appropriate for its intended use.

The term “underexpressed” as used herein refers to decreased quantity of a gene or gene product.

The terms “vector” and “expression vector” are used herein to refer to a replicon, i.e., any agent that acts as a carrier or transporter, such as a phage, plasmid, cosmid, bacmid, phage or virus, to which another genetic sequence or element (either DNA or RNA) may be attached so as to bring about the replication of the attached sequence or element and so that sequence or element can be conveyed into a host cell.

The term “vitamin” as used herein, refers to any of various organic substances essential in minute quantities to the nutrition of most animals act especially as coenzymes and precursors of coenzymes in the regulation of metabolic processes. Non-limiting examples of vitamins usable in context of the present invention include vitamin A and its analogs and derivatives: retinol, retinal, retinyl palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

The term “wild type” as used herein refers to an organism or phenotype as found in nature.

### I. Method for Identifying a Therapeutic Target for Treating a Ciliopathy

According to another aspect, the described invention provides a method for identifying a therapeutic target for treating a disease comprising a ciliopathy, the method comprising steps:

(a) providing an animal model system of the ciliopathy for testing a putative therapeutic agent, wherein the animal comprises ciliated cells;

(b) labeling the ciliated cells of the animal of step (a) with a traceable agent;

(d) administering a disruptive agent wherein the disruptive agent affects the function of at least one therapeutic target of the animal of step (a),

(e) comparing a measurable trait of the labeled ciliated cells of step (b) with a wild type animal,

(f) identifying the at least one therapeutic target of step (d) as a therapeutic target for treating a ciliopathy,

wherein the therapeutic target affects at least one trait of cilia or a dendrite extension of the ciliated neurons.

According to one embodiment, the animal is Caenorhabditis elegans.

According to another embodiment, administering step (d) further comprises associating the disruptive agent with the at least one therapeutic target. According to one embodiment, the therapeutic target is the ciliary localization-1 (cil-1) gene, the cil-1 RNA, or the CIL-1 protein. According to another embodiment, the therapeutic target is the Nephronophthisis-1 (NPHP-1) gene, the NPHP-1 RNA, or the NPHP-1 protein. According to another embodiment, the therapeutic target is the Nephronophthisis-2 (NPHP-2) gene, the NPHP-2 RNA, or the NPHP-2 protein. According to another embodiment, the therapeutic target is the Nephronophthisis-3 (NPHP-3) gene, the NPHP-3 RNA, or the NPHP-3 protein. According to another embodiment, the therapeutic target is the Nephronophthisis-4 (NPHP-4) gene, the NPHP-4 RNA or the NPHP-4 protein. According to another embodiment, the therapeutic target is the Nephronophthisis-5 (NPHP-5) gene, the NPHP-5 RNA, or the NPHP-5 protein. According to another embodiment, the therapeutic target is the Nephronophthisis-6/CEP290 (NPHP-6/CEP290) gene, the NPHP-6/CEP290 RNA, or the NPHP-6/CEP290 protein. According to another embodiment, the therapeutic target is the Nephronophthisis-7/GLIS2 (NPHP-7/GLIS2) gene, the NPHP-7/GLIS2 RNA, or the NPHP-7/GLIS2 protein. According to another embodiment, the therapeutic target is the Nephronophthisis-8/RPGRIPIL (NPHP-8/RPGRIPIL) gene, the NPHP-8/RPGRIPIL RNA, or the NPHP-8/RPGRIPIL protein. According to another embodiment, the therapeutic target is Nephronophthisis-9/NEK8 (NPHP-9/NEK8) gene, the NPHP-9/NEK8 RNA, or the NPHP-9/NEK8 protein. According to another embodiment, the therapeutic target is the polycistin-1 (lov-1) gene, the polycistin-1 RNA or the Polycistin-1 protein. According to another embodiment, the therapeutic target is the polycistin-2 (pkd-2) gene, the polycistin-2 (pkd-2) RNA, or the Polycistin-2 protein. According to another embodiment, the therapeutic target is the MKS1(Meckel-Gruber syndrome-1) gene, the MKS-1 RNA, or the MKS-1 protein. According to another embodiment, the therapeutic target is the MKS-2 (Meckel-Gruber syndrome-1) gene, the MKS-2 RNA, or the MKS-2 protein. According to another embodiment, the therapeutic target is the MKS-3 (Meckel-Gruber syndrome-3) gene, the MKS-3 RNA, or the MKS-3 protein. According to another embodiment, the therapeutic target is the Jobert syndrome (JBTS) gene, the JBTS RNA, or the JBTS protein. According to another embodiment, the therapeutic target is the SLS (Senior-Loken Syndrome) gene, the SLS RNA, or the SLS protein. According to another embodiment, the therapeutic target is the LCA (Leber Congenital Amaurosis) gene, LCA RNA, and the LCA protein.

According to another embodiment, the cell is a neuron. According to another embodiment, the cell is a renal cell. According to another embodiment, the cell is a hepatocyte.

According to another embodiment, the measurable trait is an increase or decrease of a parameter that modulates at least one function of the therapeutic target. According to some embodiments, the measurable trait is morphology. According to some embodiments, the measurable trait is connectivity. According to some embodiments, the measurable trait is movement. According to some embodiments, the measurable trait is assembly. According to some embodiments, the measurable trait is polymerization. According to some embodiments, the measurable trait is kinetics.

According to another embodiment, the disruptive agent of step (d) is a double-stranded RNA molecule. According to another embodiment, the disruptive agent of step (d) is a shRNA. According to another embodiment, the disruptive agent of step (d) is an antisense RNA. According to another embodiment, the disruptive agent of step (d) is a morpholino. According to another embodiment, the disruptive agent of step (d) is a mutagenic agent. According to another embodiment, the disruptive agent of step (d) is N-ethyl-N-nitrosourea. According to another embodiment, the disruptive agent of step (d) is ethyl methanesulfonate.

According to another embodiment, the method further comprises identifying a modulator of the disruptive agent of step (d), wherein the modulator at least partially affects the measurable trait of the therapeutic target. According to some such embodiments, the measurable trait is morphology. According to some such embodiments, the morphology is the morphology of cilia. According to some such embodiments, the measurable trait is connectivity of at least one subunit of a microtubule of the cilia. According to some such embodiments, the measurable trait is movement of at least one subunit of a microtubule. According to some such embodiments, the measurable trait is assembly of at least one subunit of a microtubule of the cilia. According to some such embodiments, the measurable trait is polymerization. According to some such embodiments, the measurable trait is kinetics of assembly of a microtubule of the cilia. According to some such embodiments, the measurable trait is kinetics of dissembly of a microtubule of the cilia. According to another embodiment, the measurable trait is translocation of an endosome comprising a protein in cilia.

According to another embodiment, the method further comprises administering an modulator of the disruptive agent of step (d) to a patient suffering from a ciliopathy, wherein the modulator of the disruptive agent at least partially affects the measurable trait of the therapeutic target by increasing the activity of the measurable trait. According to another embodiment, the method further comprises administering an modulator of the disruptive agent of step (d) to a patient suffering from a ciliopathy, wherein the modulator of the disruptive agent at least partially affects the measurable trait of the therapeutic target by decreasing the activity of the measurable trait.

According to another embodiment, labeling step (b) is performed subsequent to step (d).

According to one embodiment, the traceable agent is a lipophilic dye. According to another embodiment, the traceable agent is a fluorescence protein. According to some such embodiments, the fluorescence protein is green fluorescent protein.

According to another embodiment, the ciliopathy is a cil-1-dependent ciliopathy.

According to one embodiment, the ciliopathy is Joubert Syndrome.

According to another embodiment, the ciliopathy is a liver disease. According to another embodiment, the ciliopathy is congenital hepatic fibrosis/Caroli Syndrome. According to another embodiment, the ciliopathy is polycystic liver disease.

According to another embodiment, the ciliopathy is a kidney disease. According to another embodiment, the ciliopathy is an autosomal dominant polycystic kidney disease (ADPKD). According to another embodiment, the ciliopathy is an autosomal recessive polycystic kidney disease (ADPKD). According to another embodiment, the ciliopathy is nephronophthisis (NPHP). According to another embodiment, the ciliopathy is Bardet-Biedhl Syndrome (BBS). According to another embodiment, the ciliopathy is glomerulocystic kidney disease (GCKD). According to another embodiment, the ciliopathy is cystic dysplastic kidneys (CDK). According to another embodiment, the ciliopathy is multicystic dysplastic kidneys (MCDK). According to another embodiment, the ciliopathy is medullary sponge kidney (MSK).

Unless defined otherwise, all technical and scientific terms used herein have the same meaning as commonly understood by one of ordinary skill in the art to which this invention belongs. Although any methods and materials similar or equivalent to those described herein can also be used in the practice or testing of the described invention, the preferred methods and materials are now described. All publications mentioned herein are incorporated herein by reference to disclose and describe the methods and/or materials in connection with the publications are cited.

It must also be noted that as used herein and in the appended claims, the singular forms “a,” “and” and “the” include plural referents unless the context clearly dictates otherwise. All technical and scientific terms used herein have the same meaning.

The publications discussed herein are provided solely for their disclosure prior to the filing date of the present application. Nothing herein is to be construed as an admission that the described invention is not entitled to antedate such publication by virtue of prior invention. Further, the dates of publication provided may be different from the actual publication dates which may need to be independently confirmed.

It should be understood by those skilled in the art that various changes may be made and equivalents may be substituted without departing from the true spirit and scope of the Invention. In addition, many modifications may be made to adapt a particular situation, material, composition of matter, process, process step or steps, to the objective, spirit and scope of the described invention. All such modifications are intended to be within the scope of the claims appended hereto.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Caenorhabditis elegans cil-1 Assay

### I. Overview

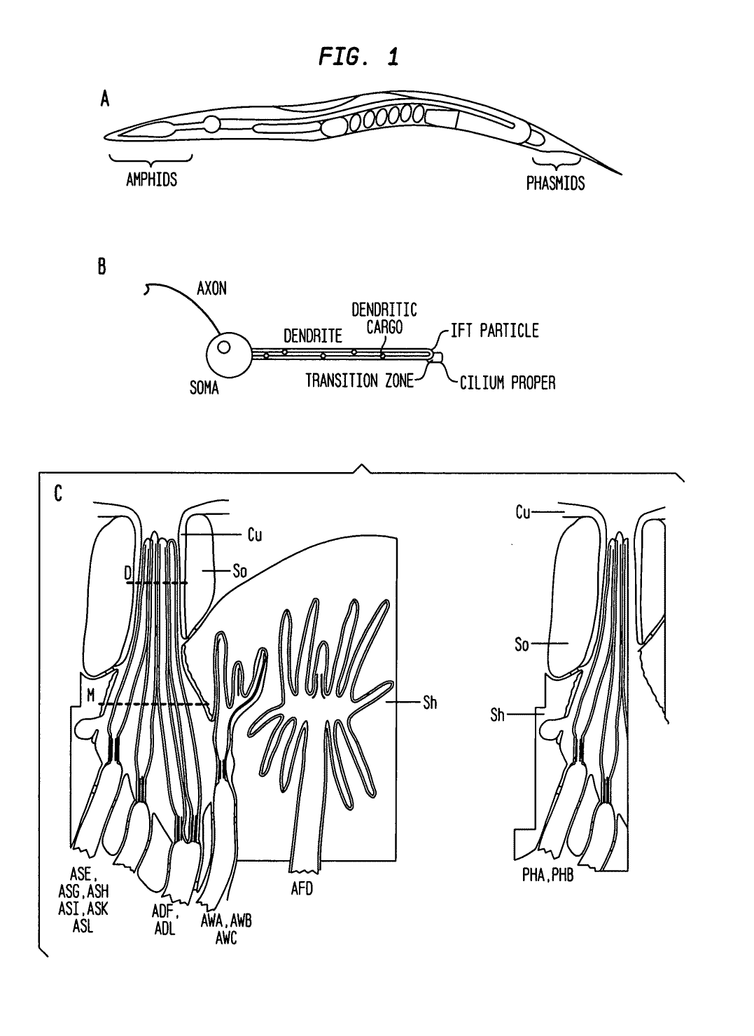

Caenorhabditis elegans is a useful in vivo model system for identifying genes and assessing potential therapeutic compounds for treating diseases caused by ciliopathy. Although C. elegans has no kidney, it possesses ciliated neurons located in three areas: the amphids (head), phasmids (tail), and male specific (head, and tail), which allows the examination of genes or compounds affecting cilial function in vivo (FIG. 1).

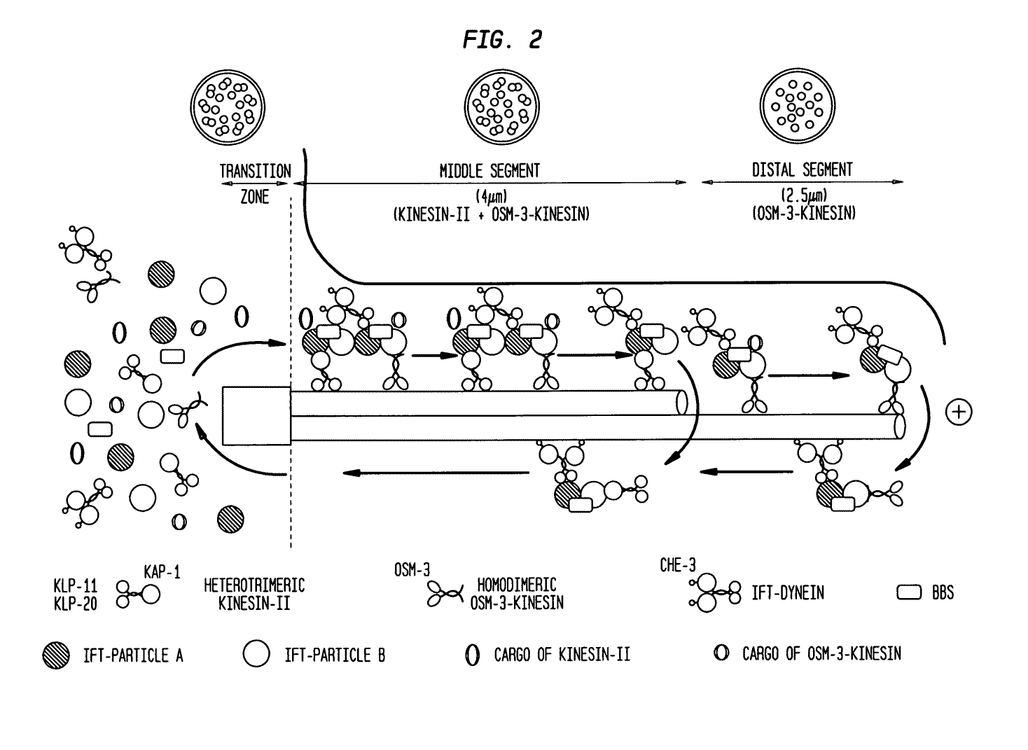

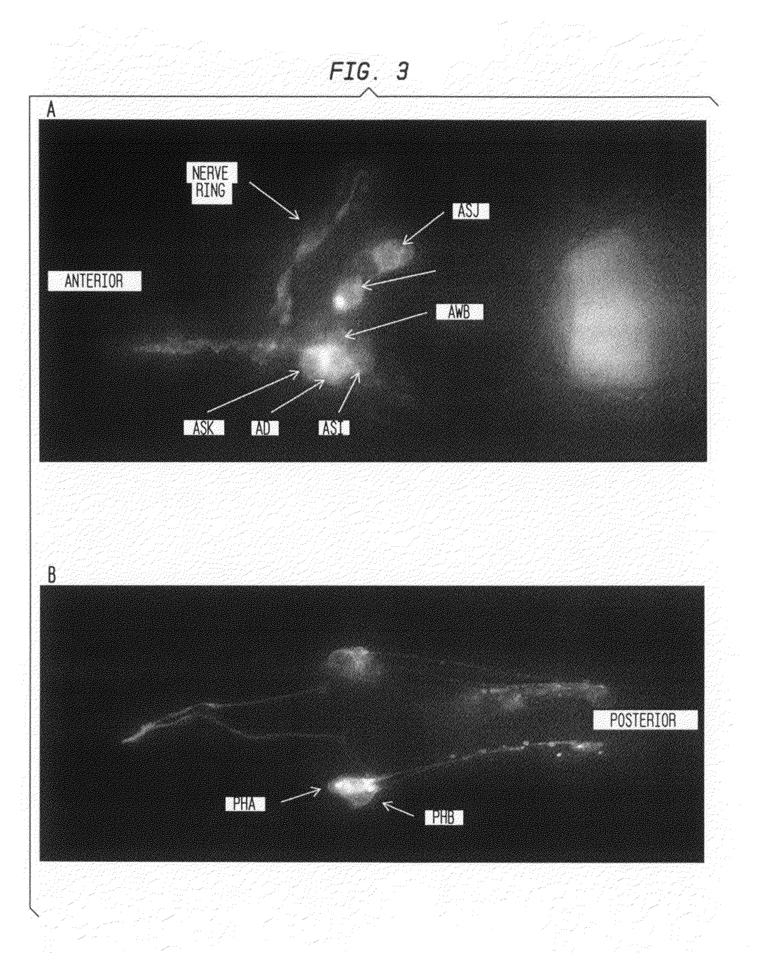

Structurally speaking, at the base of the cilium in the transition zone (TZ) lies a large complex of proteins involved in (i) the targeting and docking of the basal body to membrane, (ii) ciliogenesis, (iii) selectively filtering components entering the ciliary compartment, and (iv) loading cargo onto intraflagella transport (IFT) particles. Intraflagella transport (IFT) is the movement of ciliary components along the axoneme. Axoneme is a bundle of nine microtubules forming the internal scaffolding of a cilium, with two extra central microtubules connecting the others if the cilium is motile. There are three canonical motor proteins, which transport cargoes in and out of the cilium: kinesin-II and OSM-3 travel anterogradely, and dynein travels retrogradely. The IFT complex is composed of polypeptide subunits, IFT-A and IFT-B. OSM-5 and OSM-6 are components of IFT-B. (FIG. 2)

With respect to genes involved in cilia structure and function, a large number of the cytoplasmic proteins expressed in cilia in mammalian system are highly conserved in C. elegans. For example, the C. elegans genome encodes the homologs of genes encoding proteins involved in cilia structure and function including homologs of six NPHP genes (nphp-1, nphp-2, nphp-4, nphp-8/mks-5, nekl-1/-2, nphp-11/mks-3) and four MKS genes (mks-1, mks-3, mks-5, mks-6). These genes are all known to localize to the cilia base or the proximal segment.

Therefore, the described invention sought to identify genes or compounds which affects cilia structure and function by labeling the ciliated neurons, for example, with lipophilic dye or GFP expression, and then by examining the cilia phenotypes upon applying a candidate compound or manipulating gene/protein functions, including mutagenesis, RNA interference, and morpholino applications. Based upon the assay described below, two genes (i.e., cil-1 and nphp-2), involved in ciliopathy and the mechanism by which the proteins encoded by those genes regulate cilial function have been identified. Since these genes have been implicated in many human ciliopathy, the described invention further provides methods for treating ciliopathy caused by mutation of the cil-1 or nphp-2 genes by modulating the activity of CIL-1 protein.

### II. Methodologies: Methods, Dye Filling and Scoring Methodologies

### 1. Experimental Protocols

1.1 Caenorhabditis elegans Strains and Genetics

Nematodes were raised under standard conditions. “WT (wild type)” or “WT control” refers to him-5(e1490)V for high incidence of males. Transgenic animals were generated by germline injections. The recessive mutation my15 was isolated in a genetic screen. cil-1 (my15) was outcrossed six times. For deficiency tests, MT3022 (nDf20/sma-2(e502) unc-32(e189)III) heat-shock induced males were crossed with unc-32 my15 III; pkd-2(sy606) myIs1 [PKD-2::GFP; Pcoelomocyte::GFP] IV; him-5(e1490) V. Non-Unc F1 males (unc-32 my15/nDf20 III; pkd-2/+ myIs1/+ IV; him-5/+ V were scored for PKD-2::GFP localization. Non-Unc F1 hermaphrodites were examined for self-fertility. For SNP mapping, dpy-17 my15 unc-32 III; pkd-2 myIs1 IV; him-5 V (Unc Dpy) hermaphrodites were crossed with males from CB4856 Hawaiian strain. In F2s, either NonUnc Dpy or Unc NonDpy recombinant hermaphrodites were singled out to score Cil (in F3 males) and Spe (F2 semi-sterility) phenotypes.

A combination of three point and single nucleotide polymorphism (SNP) mapping placed the cil-1 locus between snp_C14B9 and snp_C02F5 on the linkage group III. my15/my15 and my15/nDf20 are phenotypically indistinguishable in Cil and Spe phenotypes, indicating that my15 is a null or reduction of function allele.

1.2. Molecular Cloning

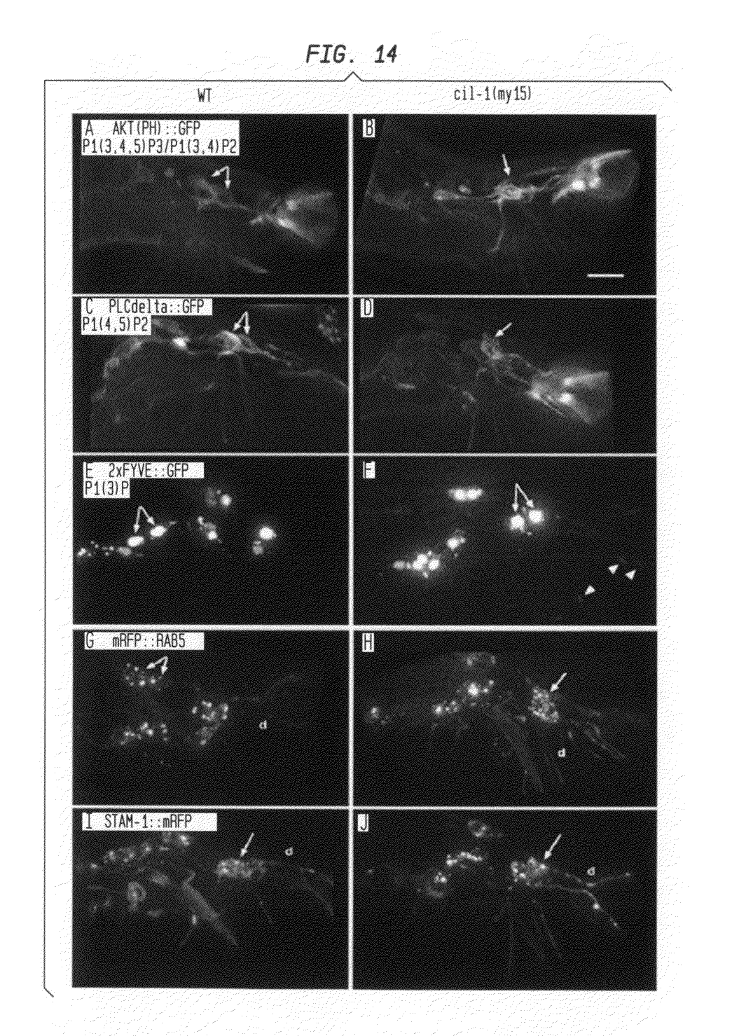

Restriction fragment and Gateway cloning (Invitrogen) methods were used to generate plasmids for fluorescent protein fused markers, which include GFP (green fluorescent protein), Mrfp (monomeric) and tdtomato (tandem dimer tomato) (Clontech). For rescuing experiments, a series of PCR amplified genomic fragments along with the dominant rol-6 marker at 60 ng/ul each were injected individually or in pools into WT animal and resulting transgenic lines were crossed into my15 animals to score rescue of PKD-2::GFP mislocalization (Cil phenotype) and/or the spermatogenesis-defective (Spe phenotype) (FIG. 13). The genomic coordinates for fragments mentioned in FIG. 10 are; 8150K: III: 8,146,448 to 8,155,624, 8160K: III: 8,154,210 to 8,164,811, 8160K-1: III: 8,158,952 to 8,164,811. To create PCR-SOEd products, the bath-42 promoter was amplified by 5′ACTCACTGCTTTCAAGTTGCTGA3′ [SEQ ID NO: 1] and 5′AGTCGATCGATGCCTCGATACCTCTGTTGATAAGTTGAGCGAG3′ [SEQ ID NO: 2]. The cil-1 genomic region was amplified by

5′ TCGAGGCATCGATCGACTATAACAATGGATTGGAAAATAACAATATTC3′ [SEQ ID NO: 3] and 5′TGCAAGACTATTAGGGAGTG3′ [SEQ ID NO: 4]. The linker is italicized. The my15 lesion was identified by sequencing using 5′AGGAGCCGTACTTACATGG3′ [SEQ ID NO: 5] (sense) and 5′AATTTGCCGCGTCGAGAC3′ [SEQ ID NO: 6] (antisense).

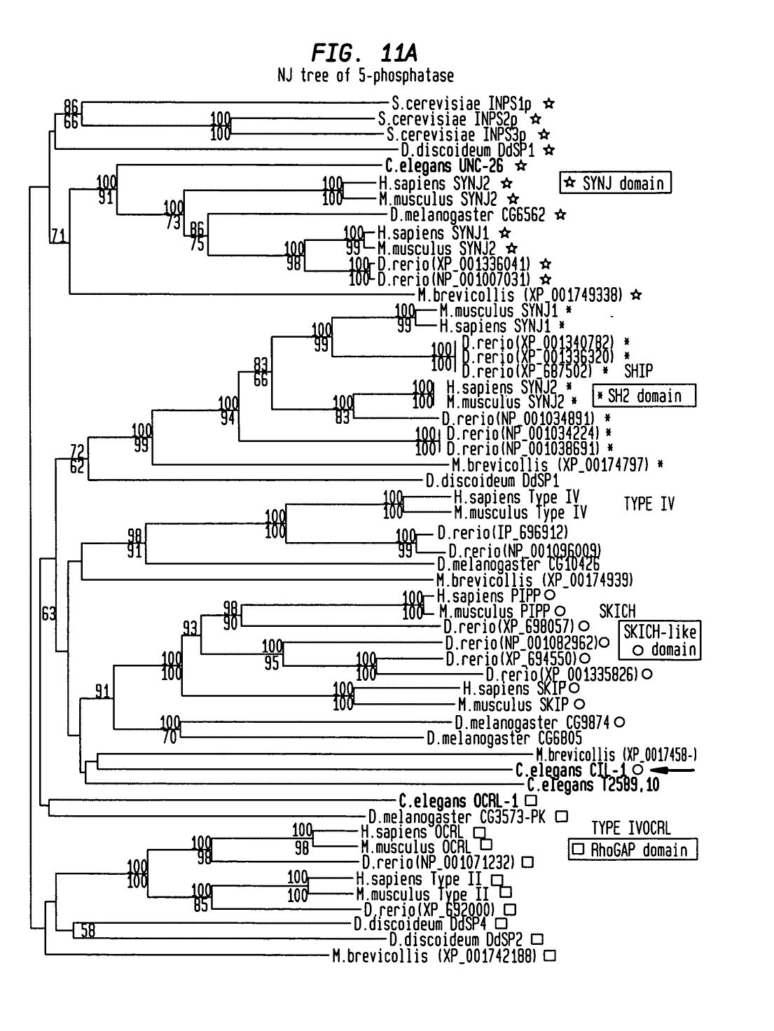

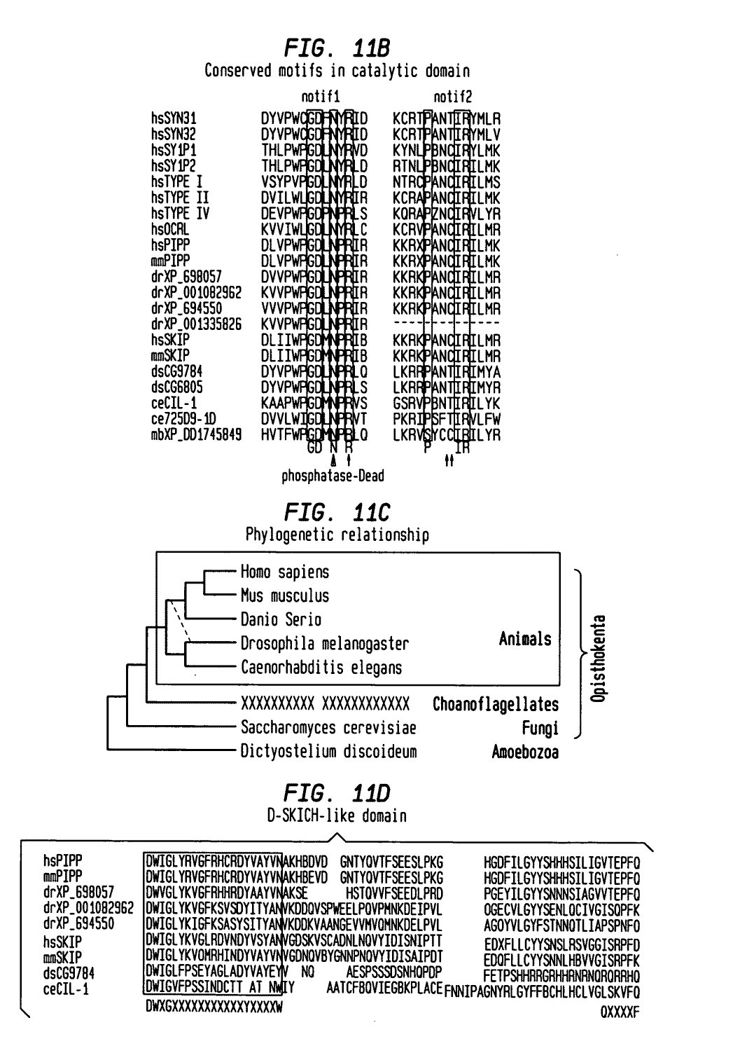

To determine cil-1 expression pattern, a 2.2 or 1.5 kb 5′UTR of bath-42, the gene upstream of cil-1 in the operon, was used to drive GFP expression. Pbath-42::GFP promoter fusions are broadly expressed in a highly mosaic manner including the intestine, pharynx, unidentified head and male tail neurons, body wall muscles, and hypodermis, but are not overlapping with pkd-2 (FIG. 11). Moreover, Pbath-42::CIL-1::GFP expression does not rescue the my15 Cil phenotype (data not shown), while a cil-1(+) genomic fragment containing the C50G3.7 250 bp 5′UTR, genomic region, and 200 bp 3′UTR partially rescues the my15 Cil phenotype (FIG. 3B). These data indicate that 5′ UTR and 3′UTR of cil-1, and the 5′UTR of the operon coordinate cil-1 expression.

1.3. Microscopy

Epifluorescence and Nomarski analyses were performed on a Zeiss Axioplan2 imaging system equipped with 10×, 63× (NA 1.4), 100× (NA 1.4) objectives and a Photometrics Cascade 512B CCD camera using Metamorph software. 3D deconvolution of Z-stacks of epifluorescent image were performed by AutoQuantX program (MediaCybernetics). Confocal images were collected using a 510 LSM META ZEISS. Optical sections were collected between 0.4-1.0 μm and projected as Z-series that were stored as TIFF files and manipulated using Adobe PhotoShop™. Worms were anesthetized with 1 mM levamisole, mounted on agar pads and maintained at room temperature. For time-lapse microscopy of sperm motility, Nomarski images were taken every 15 seconds for up to 15 minutes. Neuronal images shown in figures are projected epifluorescent Z-stacks after 3D deconvolution except for FIGS. 2C and 2G, which are projected Z-stacks of confocal sections. For electron microscopy, sperm were prepared as known in the art, except that the samples were embedded in LX112 (Ladd Research Industries Inc., Burlington, Va.).

1.4. Behavioral Assays

Male behavioral assays (response and location of vulva efficiency) were performed as known in the art. At least 20 males per genotype were scored per assay. Response efficiency reflects the percentage of the males that responded to the hermaphrodites within 4 minutes. Location of vulva efficiency was calculated by successful vulva location divided by the total number of vulva encounters for each male. Spicule insertion efficiency was determined by the fraction of males that successfully insert the spicule during 15 minutes of observation with 3-day old unc-31 hermaphrodites. Statistical analyses were performed by t-tests with two-tailed p-value. Mating efficiency was performed as described. For sperm tracking assays, we isolated L4 males for 2 days and soaked in 1 μg/ml MitoTracker Red CMXRos (Invitrogen) in M9 solution for 30 minutes. After recovery on NGM plates, four males and three unc-31 hermaphrodites are placed on each plate. For brood size, a single L4 hermaphrodite was isolated to a plate and kept at 20 degrees. The number of F1s was counted until the hermaphrodite stopped laying eggs. Dye filling, diacetyl olfaction, and osmotic avoidance assays were performed.

1.5. Sperm Isolation and Activation

To isolate spermatids, virgin L4 males were isolated for 2-3 days in 20 degrees and cut with a 25g needle on a glass slide in Sperm Media (SM) pH 7.9 with 10 mM Dextrose. For in vitro activation, 20 ug/ml of Pronase or 100 nM monensin (for in vitro sperm crawling experiments) was added to SM as described in [21]. For wortmannin treatment, a 100× wortmannin stock solution in DMSO was added to reach the final concentration of 100 nM into SM.

1.6. Immunohistochemistry

For whole animal fixation, animals were washed free of bacteria in M9 and placed on poly-Llysine coated slides in 2-3 ul of PBS pH 7.4 containing 4% paraformaldehyde for 5 minutes at room temperature. A cover slip was put on the slide, it was placed onto a metal block cooled by dry ice for 20 minutes and then the coverslip was popped off with a razor blade. The slide was transferred to a containing −20° methanol for 15 minutes, rehydrated in PBS and either

antibody or DAPI staining was performed [22]. For isolated sperm immunochemistry, before and after adding in vitro activator, a 4× formaldehyde solution was added to a final concentration of 4%, followed by conventional immunohistochemistry methods.

Phylogenetic analysis of 5-phosphatases PI 5-phosphatase sequences obtained from GenBank or Wormbase included three vertebrates (Homo sapiens, Mus musculus, Danio rerio), two invertebrates (Drosophila melanogaster, Caenorhabditis elegans), and three unicellular eukaryotes (Monosiga brevicollis, Saccharomyces cerevisiae, Dictyostelium discoideum). 5-phosphatase catalytic domains were aligned using MacClade ver. 4.08. A total of 261 unambiguously aligned amino acid positions were included in the final alignment. Due to highly derived sequence nature, the Type I-like inositol 5-phosphatase sequences were excluded in phylogenetic analyses. Phylogenetic analysis was performed using PAUP* ver. 4.0 Neighbor joining and maximum parsimony trees were constructed and bootstrap values were obtained by 100 re-samplings.

### 2. Dye Filling

Dye filling utilizes a lipophilic fluorescent dye that is taken up by exposed neurons by an unknown mechanism. Presence of morphologically aberrant cilia, in addition to dendritic extension errors (Dex), prevents dye uptake and neuronal staining. This allows the examination of genes involved in amphid (head) and phasmid (tail) ciliogenesis. Specifically, hermaphrodites are staged by picking 8-10 animals to a seeded NGM plate. After three days, the worms are washed off with M9, then rinsed three times. M9 is added to total 700 μL, and DiI is added to a concentration of 40 μg/mL. Foil covered tubes are placed on 1 Hz shaker for 1 hour, then washed with M9 three times, and allowed to crawl out on a lawn for 30-60 minutes.

### 3. Scoring

3.1. Amphid (Head)

In the wild type (WT) hermaphrodite (him-5), twelve amphid neurons take up dye: left and right ASK, ADL, ASI, AWB, ASH, ASJ. (FIG. 3A). Neurons are scored individually in each animal, and then averaged together to yield Fraction Amphids (head) Dye Filling. There were no obvious trends in cell type specific dye filling defects. At least two replicates of 20 animals each were assayed.

3.2. Phasmid (Tail)

In the WT hermaphrodite, four phasmid neurons take up dye: left and right PHA and PHB. (FIG. 3B). Neurons are scored in left-right pairs, and then averaged together to yield Fraction phasmids Dye Filling. Two to three replicates of 20-30 animals each were assayed.

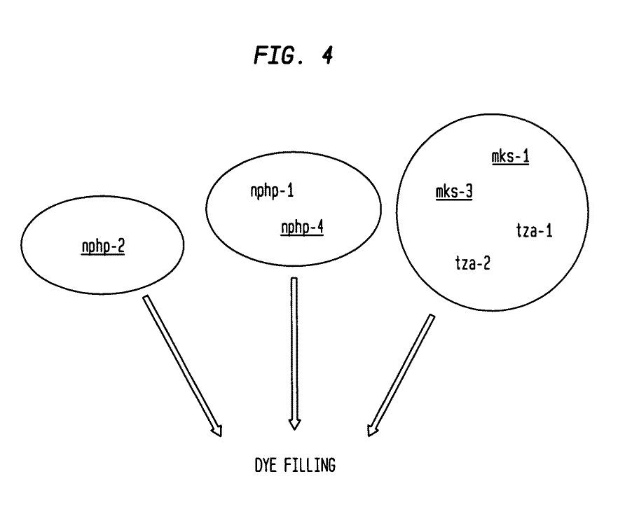

The phasmid dye filling data has allowed the grouping of genes with TZ localizing products into three distinct pathways. Deletion of two genes within a pathway, as in nphp-1; nphp-4 and mks-1; mks-3 double mutants, yields animals with WT dye filling. Deletion of two genes in any of the three pathways results in a synthetic dye filling (SynDyf) phenotype. (FIG. 4)

To better characterize the three pathway model, genes of each arm were selected, and all possible double mutant combinations were built. Determining in which arm a new gene of interest acts would then be as simple as crossing it with each of these “archetypal” genes. All nphp-2 double mutants except for nphp-2 nphp-4 are similar to WT. This implies a different mechanism of action for nphp-2 in each cell type. Without being limited by theory, possibilities include either a role in the mks branch of the pathway, or a role modulating both nphp and mks pathways in phasmids, but modulating only the nphp pathway in the amphids. This goes counter to the idea that amphids and phasmids can be grouped together. Instead amphid and phasmid data should be combined carefully.

### III. Results

### (A). Ciliary Phenotypes can be Dissimilar in Amphids and Phasmids

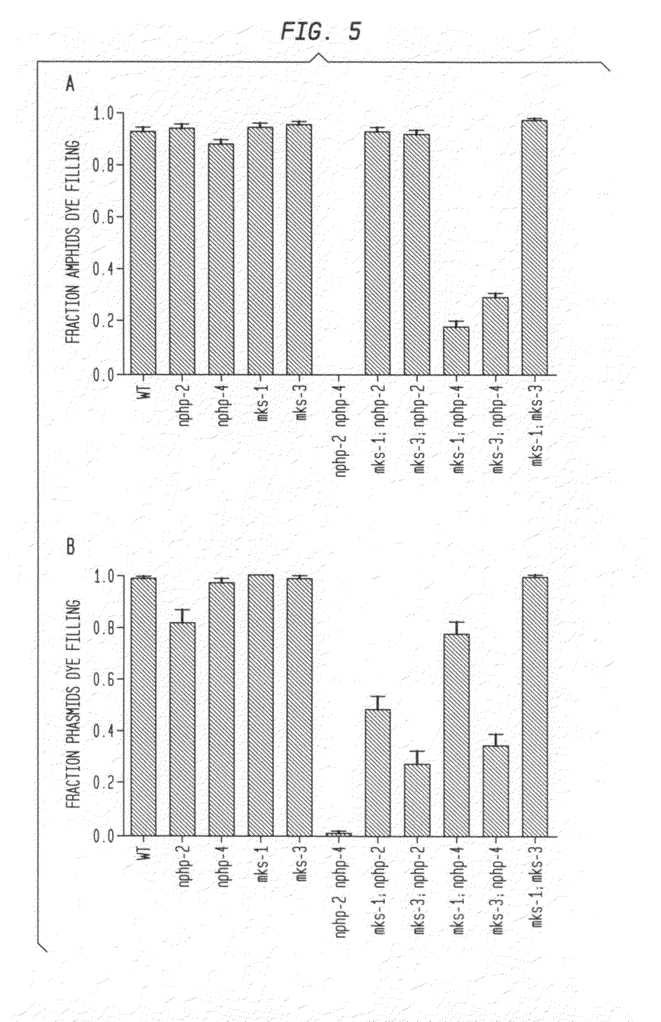

Examination of all double mutant combinations between genes from each pathway arm reveals phasmid dye filling defects of varying degrees in all double mutants except mks-1; mks-3, as expected. mks-3; nphp-X double mutants have more severe phenotypes than mks-1; nphp-X double mutants. (FIG. 5). All nphp-2 double mutants except for nphp-2 nphp-4 are similar to WT. This implies a different mechanism of action for nphp-2 in each cell type. Without being limited by theory, possibilities include either a role in the mks branch of the pathway, or a role modulating both nphp and mks pathways in phasmids, but modulating only the nphp pathway in the amphids. This goes counter to the idea that amphids and phasmids can be grouped together. Instead amphid and phasmid data should be combined carefully.

(B). mks-6 is in the Same Pathway as other mks Genes

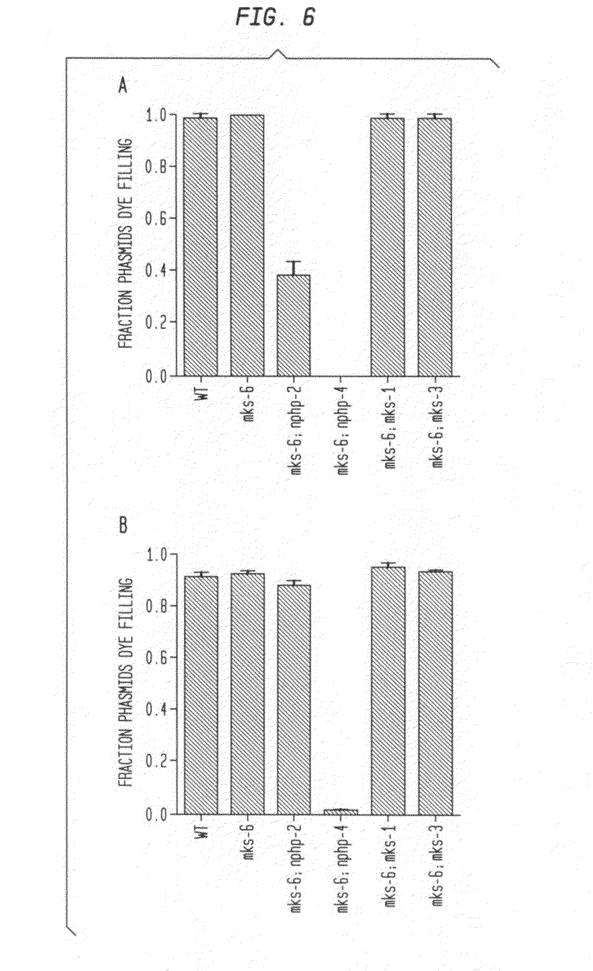

In phasmids, mks-6; nphp-4 shows severe Dyf as strong as nphp-2 nphp-4. mks-6; nphp-2 double mutants have an intermediate phasmid Dyf phenotype, but a WT phenotype in the amphids. Within the phasmids, mks-6 falls into the same pathway as mks-3 and the B9 genes. The mks-6; nphp-2 phenotype fits in with the data from the previous panel showing a more important role for nphp-2 in phasmid cilia. (FIG. 6)

(C). The Phosphoinositide 5-phosphatase cil-1 is SynDyf with nphp-2

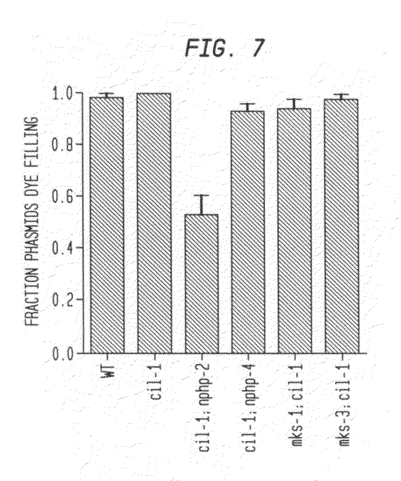

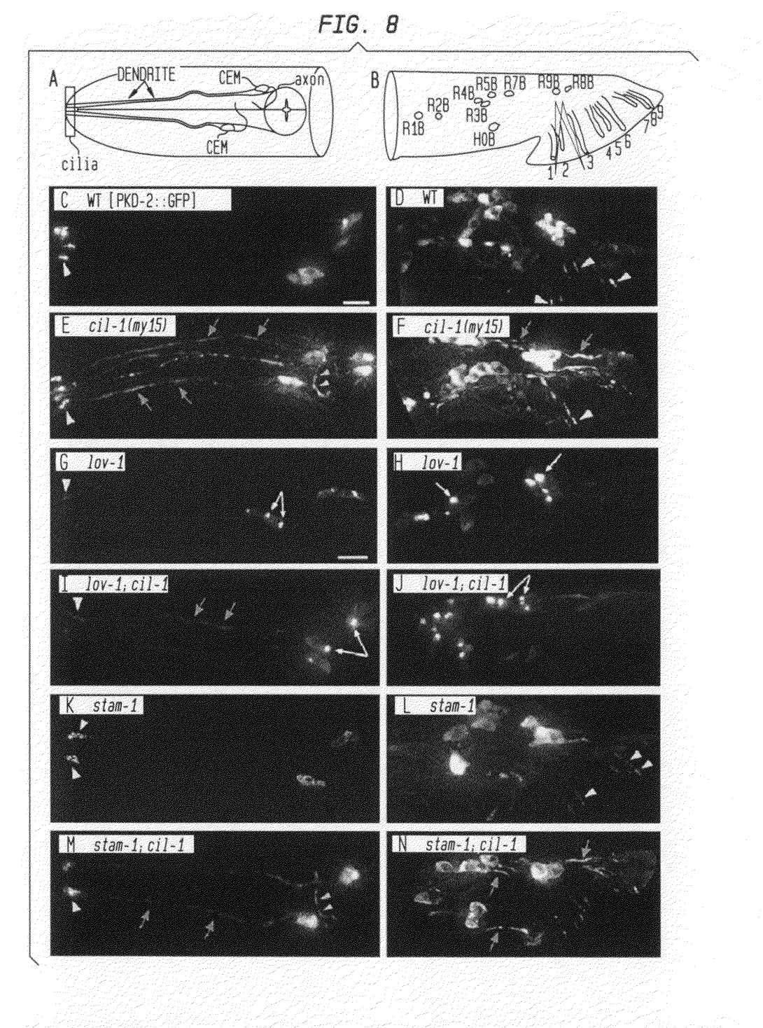

cil-1, a homolog of the ciliopathy gene INPP5E, is mutated in individuals with the ciliopathy Joubert Syndrome (JBTS1) and exhibits synthetic dye filling (SynDyf) phenotype with nphp-2 in the phasmids. (FIG. 7). This relates to a connection between Wnt modulation of PIP signaling—the disheveled mediated conversion of PI→PI(4)P→PI(4,5)P→PI(3,4,5). In zebrafish, the nphp-2 homolog Inversin antagonizes cytoplasmic Dvl, decreasing PI(3,4,5)P levels. cil-1 can dephosphorylate PI(3,4,5) to PI(3,4), and also decrease PI(3,4,5) levels.

### (D). CIL-1 is Required for LOV-1 and PKD-2 Localization

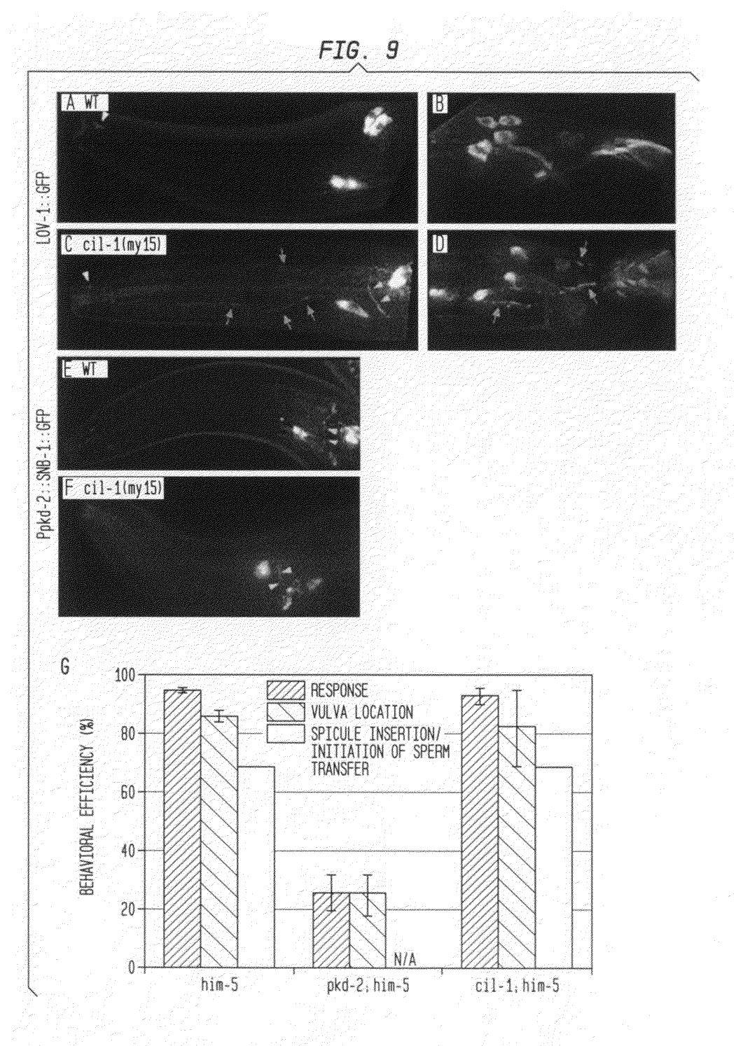

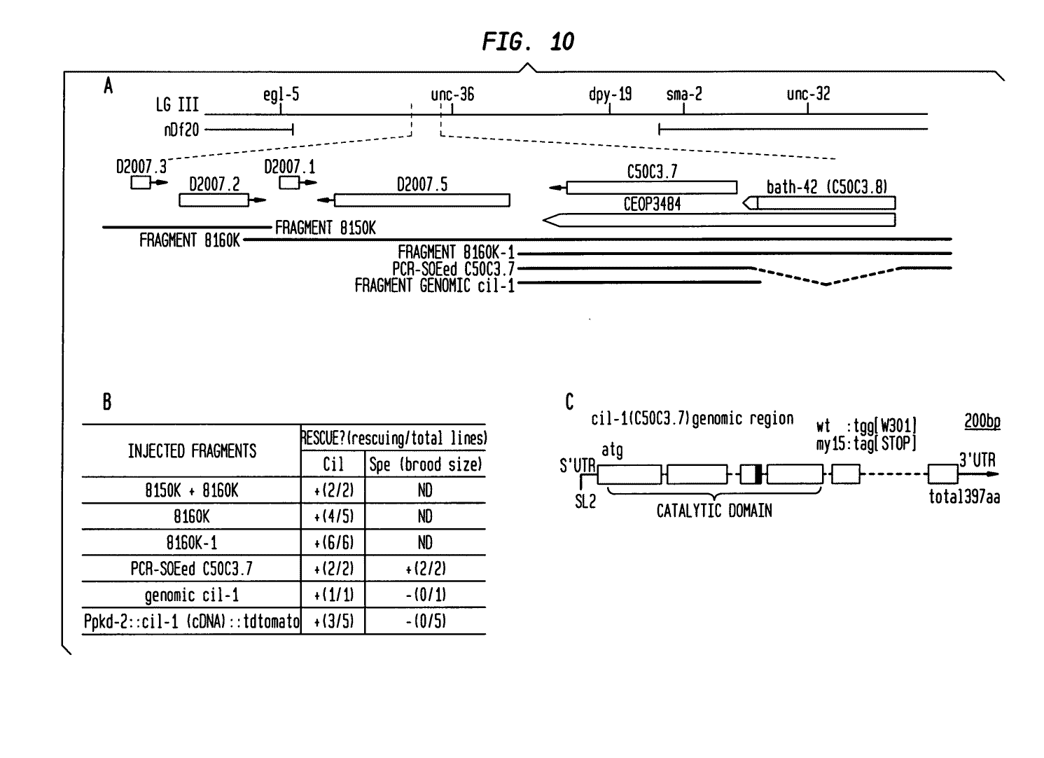

In wild-type (WT), PKD-2::GFP (i.e., PKD-2 tagged with GFP) localizes to cell bodies and ciliary endings of 21 male-specific neurons in the head (cephalic CEMs) and tail (ray RnBs and hook HOB) (FIGS. 8, A-D). In my15 mutants (a mutant allele of cil-1), PKD-2::GFP is distributed throughout these male-specific neurons including dendrites, axons, cell bodies, and cilia (FIGS. 8, E and F). cil-1 is not required for neuronal cell fate or development of pkd-2-expressing neurons as judged by reporters including transcriptional and soluble Ppkd-2::GFP, OSM-6::GFP (data not shown), Ppkd-2:SNB-1:GFP (FIG. 9), and fluorescent-protein-tagged PI-markers (data not shown). Ppkd-2::GFP and endogenous pkd-2 mRNA levels are unaltered in my15 animals as judged by qRT-PCR (data not shown), indicating that my15 may affect PKD-2 protein expression or stability but not gene expression. In WT and my15 dendrites, small PKD-2::GFP particles move bidirectionally, indicating that a pool of PKD-2::GFP is properly trafficked in my15 dendrites. Moreover, PKD-2::GFP distribution in the my15 ciliary region appears normal, despite abnormally increased dendritic and axonal distributions (FIGS. 8E and 8F).

The distribution of additional GFP-tagged ciliary proteins was examined, including functional LOV-1::GFP; a TRP-vanilloid, OSM-9; a G protein-coupled receptor (GPCR), ODR-10; an IFT B-complex polypeptide, OSM-6; and an IFT modulator, BBS-5. Only LOV-1::GFP is abnormally distributed to dendritic and axonal processes in my15 sensory neurons (FIGS. 9C and 9D). The localization of the presynaptic marker synaptobrevin Ppkd-2::SNB-1::GFP was also examined. In WT and my15 males, SNB-1::GFP labels presynaptic puncta along axonal processes (FIGS. 9E and 9F), indicating that cil-1 does not grossly affect axonal targeting. my15 mutants are normal in lipophilic DiI dye filling of ciliated sensory neurons, chemotaxis to diacetyl, dauer formation, and osmotic avoidance (data not shown). Therefore, it was concluded that cil-1 is specifically required for localization of the TRPP complex but not for general receptor trafficking, ciliogenesis, neuronal polarization, or sensation.

lov-1, pkd-2, and a subset of Cil mutants are response and location of vulva (Lov) defective during male mating. my15 males exhibit normal response and vulva location behaviors (FIG. 9), which is explained by the presence of PKD-2::GFP in cilia. cil-1 double- or triple-homozygous or transheterozygous mutants with pkd-2 and lov-1 did not alter male mating efficiency, indicating that cil-1 is not a genetic modifier of the C. elegans TRPP genes (data not shown). Although mating behaviors appear normal, my15 males are largely infertile and produce few offspring because of a sperm (Spe) defect.

(E). cil-1 Acts Between lov-1 and stam-1 in RnB Ray Neurons

Previous study showed that TRPP complex formation is important for trafficking. In a lov-1 mutant, PKD-2::GFP forms aggregates in cell bodies and localizes to cilia at a reduced level (FIGS. 8G and 8H). In lov-1; cil-1 CEM neurons (FIG. 81), the PKD-2::GFP localization phenotype is additive: bright aggregates in the cell bodies (lov-1 phenotype) and mislocalization to the dendrites and axons (cil-1 phenotype), albeit at reduced levels. In lov-1; cil-1 RnB neurons (FIG. 1J), the PKD-2::GFP localization phenotype resembles that of lov-1 but not cil-1: aggregates in the cell body and absence from dendritic and ciliary compartments. This strict requirement of lov-1 in RnB as compared to CEM neurons has been previously shown for PKD-2 ciliary targeting. The basis of this cell-type specificity is unknown, but may be due to differences in protein trafficking mechanisms or structural differences between CEM and RnB neurons.

PKD-2 ciliary abundance is tightly controlled. STAM and Hrs mediate PKD-2 and LOV-1 downregulation via transport from early endosomes to endosomal sorting complexes (ESCRT). Reducing stam-1 or hgrs-1 function results in PKD-2::GFP and LOV-1::GFP accumulation at the ciliary base (FIGS. 8K and 8L). In CEM neurons of a stam-1(ok406); cil-1(my15) double mutant (FIG. 8M), the PKD-2::GFP localization phenotype is additive. In stam-1; cil-1 RnB neurons (FIG. 8N), PKD-2::GFP is distributed evenly throughout, similar to cil-1 single mutants. Therefore, it is concluded that, in RnB neurons, cil-1 acts after lov-1 but before stam-1. In CEMs, the lov-1; cil-1 and cil-1; stam-1 phenotype is complex, making it difficult to place lov-1, cil-1, and stam-1 in a linear or parallel pathway.

(F). cil-1 Encodes a Phosphoinositide 5-Phosphatase that Acts Cell Autonomously

The smallest rescuing genomic fragment for both the Cil and Spe phenotypes (8160K-1, FIGS. 10A and 10B) contains two genes, C50C3.7 and bath-42 in the operon CEOP3484. The bath-42(tm2360) deletion mutant is nonCil and nonSpe (data not shown). A construct containing 2.2 kb 5′UTR of the bath-42 and the C50C3.7 genomic region including the 3′UTR rescues both Cil and Spe phenotypes of my15 (PCR-SOEed C50C3.7, FIGS. 10A and 10B). Sequencing analysis of my15 genomic DNA identified a G→A transition that converts Trp301(TGG) to a stop codon (TAG) in the 5th exon of C50C3.7 (FIG. 10A). From both WT and my15 cDNA pools, reverse transcriptase PCR (RT-PCR) identified two cil-1 cDNAs (long C50C3.7a and short C50C3.7b). The short form (C50C3.7b) is generated by alternative splicing within the third intron, introducing a stop codon at the 124th amino acid before the my15-induced lesion. Ppkd-2::CIL-1a(C50C3.7a)::tdTomato fully rescues the cil-1(my15) Cil but not the Spe phenotype (FIGS. 10A and 10B), which is not surprising given that pkd-2 is not expressed in sperm. In contrast, expression of C50C3.7a with an intestinal promoter fails to rescue the Cil phenotype (data not shown). Rescue of the cil-1(my15) Spe phenotype by using cell-type-specific promoters was not performed, because transgenes are often silenced in the germline. Based on these data, it is concluded that C50C3.7 is the gene mutated in cil-1(my15) animals and that CIL-1 acts autonomously in male-specific neurons to control TRPP localization.

cil-1 encodes a phosphoinositide (PI) 5-phosphatase (referred to as 5-phosphatase), which removes the D-5 phosphate from the inositol ring of membrane-associated PI or soluble inositol phosphates. The 5-phosphatase family comprises ten mammalian, four yeast, and five C. elegans enzymes. Phylogenetic analysis of the 5-phosphatase catalytic domain and/or of the presence or absence of adjacent domains reveals that CIL-1 is closely associated with two mammalian 5-phosphatases: SKIP (skeletal muscle and kidney-enriched inositol phosphatase) and PIPP (proline-rich inositol polyphosphate phosphatase). CIL-1, SKIP, and PIPP belong to the SKICH (SKIP carboxyl homology) subfamily, which contains a C-terminal SKICH-like domain (FIGS. 11A and 11D) and mediates protein localization.

The C. elegans genome encodes five 5-phosphatase genes: ipp-5, unc-26, ocrl-1, cil-1/C50C3.7, and T25B9.10. ipp-5 (type I) negatively regulates ovulation by inhibiting inositol 1,4,5-triphosphate (IP3) signaling in the spermatheca IP3 signaling also regulates mating behavior steps of turning, spicule insertion, and sperm transfer. unc-26 (synaptojanin) is required for synaptic vesicle endocytosis and recycling, with mutants exhibiting uncoordinated movements. Neither the ipp-5 nor the unc-26 mutant is Cil or Spe defective, and cil-1(my15) mutants are normal in ovulation, locomotion, male turning, spicule insertion, and sperm transfer, ruling out possible overlapping functions (data not shown).

CIL-1 contains two conserved 5-phosphatase motifs in its catalytic domain (FIG. 11B). A missense mutation was introduced in a known critical residue of 5-phosphatase motif (CIL-1N175A, FIG. 11B, arrowhead). Ppkd-2:: CIL-1N175A::tdTomato failed to rescue the my15 Cil phenotype, indicating that phosphatase catalytic activity is required for CIL-1 function.

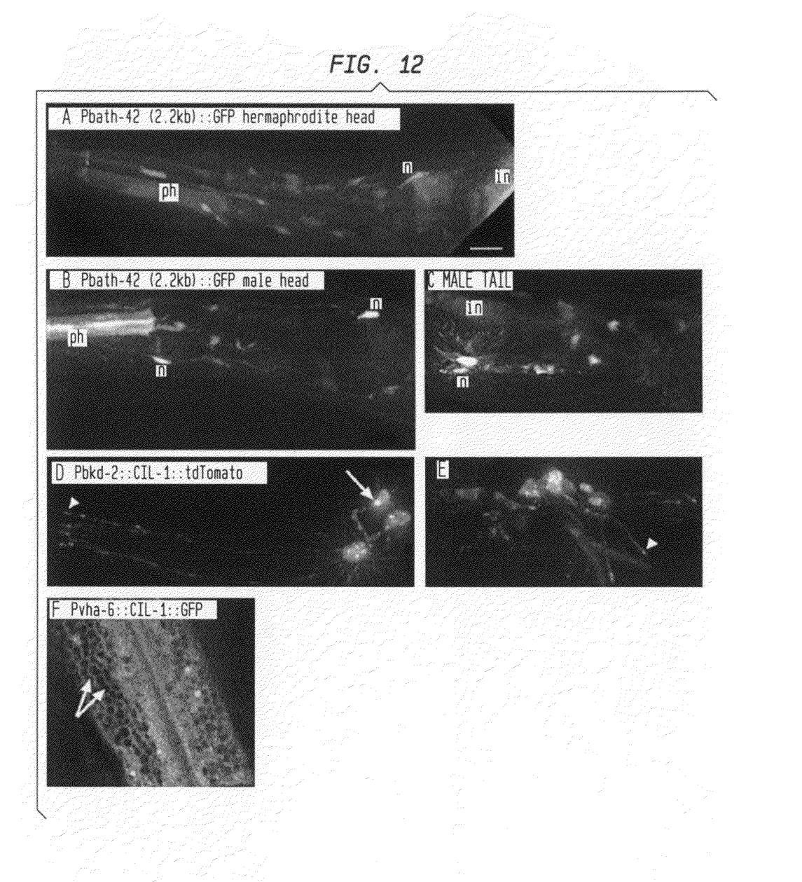

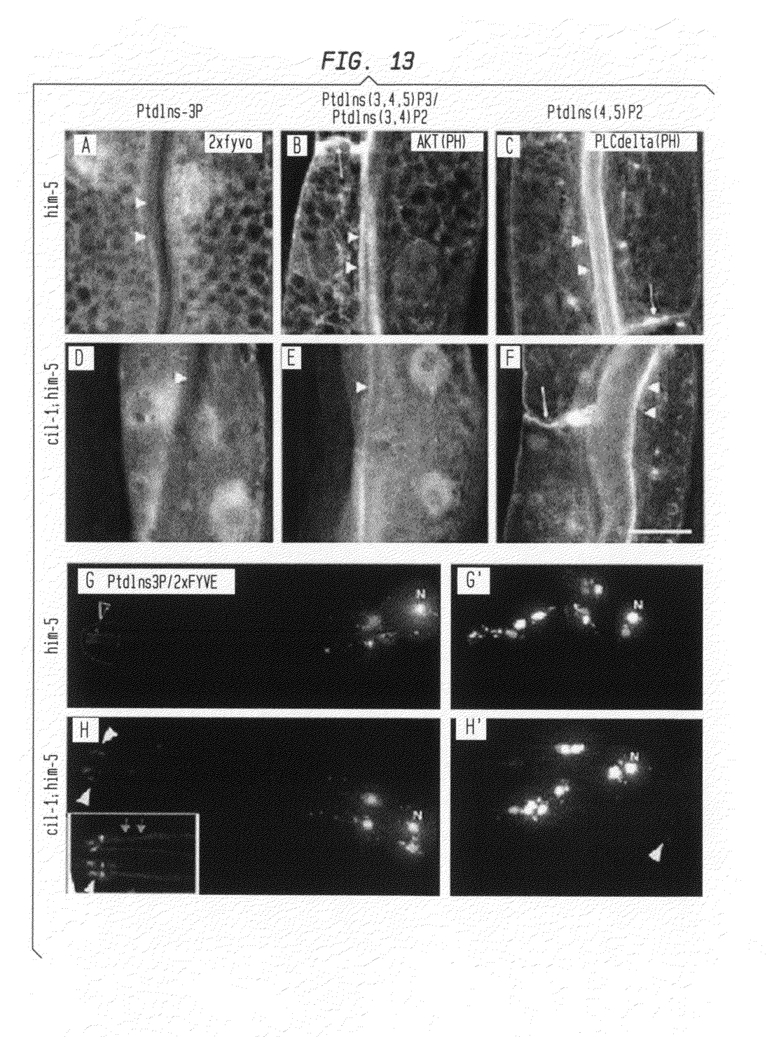

In male neurons, the rescuing Ppkd-2::CIL-1::tdTomato is distributed in cilia, dendrites, axons, cell bodies with occasional small puncta, and weakly in nuclei (FIG. 12D), and it is often visible as bright dots at ciliary bases of ray neurons (FIG. 12E), suggesting CIL-1 function in ciliary regions. In the intestine, Pvha-6::CIL-1::GFP localizes to cytoplasmic reticular structures (FIG. 12F).

### (G) CIL-1 Regulates PI(3,4)P2/PI(3,4,5)P3 and PI(3)P but Not PI(4,5)P2 Levels

Seven PI species are generated by the reversible phosphorylation and dephosphorylation. To determine what PI species are CIL-1 substrates, we expressed genetically encoded biosensors to detect changes in specific PI lipid concentrations in male-specific sensory neurons and the intestine. We observed obvious differences in PI(3)P Hrs(2×FYVE) and PI(3,4)P2/PI(3,4,5)P3 AKT(PH), but not PI(4,5)P2 (PH domain of PLC-delta) markers between WT and cil-1(my15) animals. In the WT intestine, PI(3)P is primarily found in tubulovesicular structures without any plasma membrane (PM) enrichment, similar to the CIL-1 distribution pattern (compare FIG. 13A with FIG. 12F). In the cil-1 intestine, PI(3)P is severely disrupted, with a diffuse pattern in the cytoplasm (FIG. 12D). In WT intestine, PI(3,4)P2/PI(3,4,5)P3 is found in tubulovesicular structures and enriched at the PM (FIG. 13B, arrowheads and arrow). In cil-1(my15) mutants, the PI(3,4)P2/PI(3,4,5)P3 marker labels no distinct structure, appearing diffuse in the cytoplasm with no PM enrichment. A similar pattern has been reported in C. elegans let-512/vps-34 PI 3-kinase mutants, in which PI(3)P generation is reduced. In both WT and cil-1(my15) intestine, PI(4,5)P2 is enriched at the apical PM lining the intestinal lumen as well as basolateral PM (FIGS. 13C-13F). These data indicate that CIL-1 displays in vivo phosphatase activity toward PI(3,4,5)P3 and PI(3,5)P2 in the intestine.

In male-specific sensory neurons, differences in PI(3)P distribution (FIGS. 13G and 13H) were observed, but no PI(3,4)P2/PI(3,4,5)P3 or PI(4,5)P2 markers in cil-1(my15) were observed in males (FIG. 14). In WT neurons, the PI(3)P marker is enriched in nuclei and small puncta in the cell bodies, but rarely in dendritic and ciliary regions (FIGS. 13G and 13G′), with only 1/13 animals displaying detectable expression in dendrites and cilia. In cil-1(my15), the PI(3)P marker decorates dendritic processes and cilia (16/27 animals) in addition to the enrichment in the nuclei and cell bodies (FIGS. 13H and 13H′). The PI(3)P marker is distinctly bright at cil-1(my15) ciliary bases (FIG. 13H, inset), hinting that loss of CIL-1 perturbs PI(3)P distribution in this region. These data suggest that CIL-1 displays a tissue-specific substrate preference toward PI(3,5)P2 in male sensory neurons.

Because PI(3)P localizes to early endosomes, we examined the distribution of early endosomal proteins. RAB-5 is an early endosomal protein that recruits and activates PI(3)P-generating PI 3-kinases. STAM-1 colocalizes with RAB-5 in C. elegans male-specific sensory neurons and promotes polycystin trafficking from early endosomes to the ESCRT complex. In WT and cil-1(my15) animals, RAB-5 and STAM-1 localize to small puncta in the cell bodies, axons, and dendrites (FIGS. 14G-14J), indicating that cil-1 does not modify the overall organization of early endosomes.

(H) cil-1 Mutants are Sperm Defective

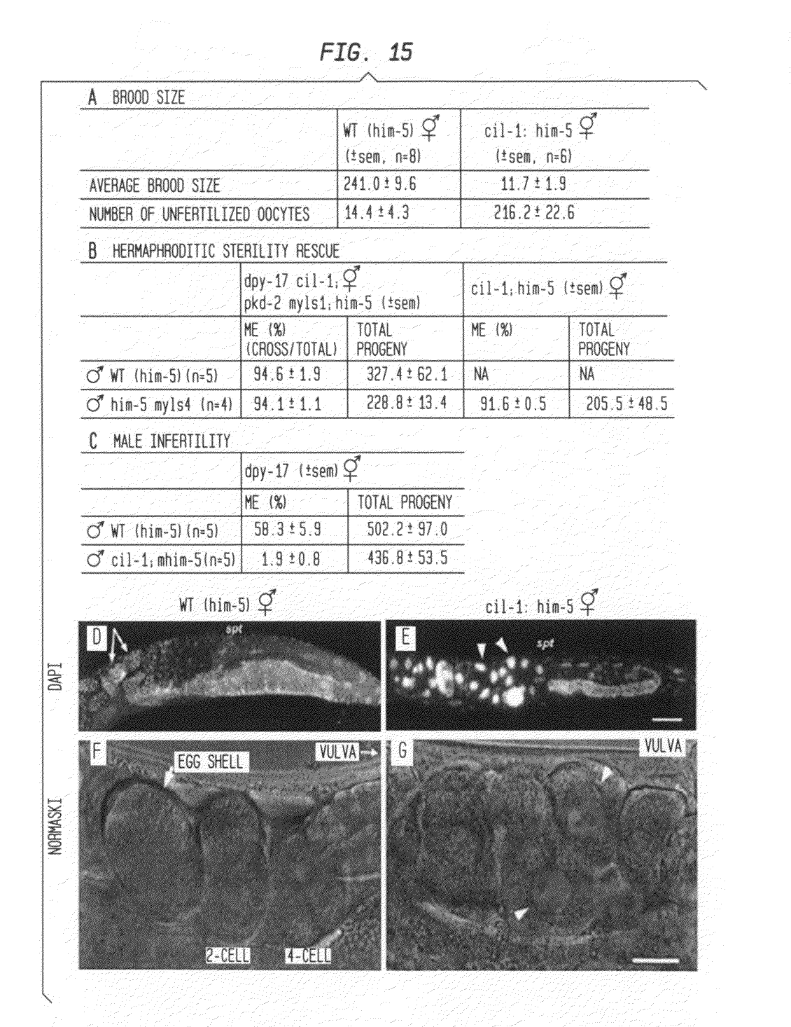

cil-1(my15) hermaphrodites exhibit a drastic reduction in brood size (4.85% of WT) (FIG. 15A), which is due to a spermatogenesis defect (Spe) based on the following observations: (1) a my15 hermaphrodite lays 200-300 unfertilized eggs, which is comparable to the number of WT fertilized eggs (FIG. 15A); (2) my15 male germline architecture and early stages of spermatogenesis (data not shown), hermaphrodite oocyte maturation, and ovulation appear normal (FIGS. 14D and 14E); (3) my15 hermaphrodites contain endomitotic cells without eggshells in the uterus (FIGS. 15F and 15G); (4) the my15 fertility defect is completely rescued when my15 hermaphrodites are mated with WT males (FIG. 15B); and (5) my15 males fail to sire cross progeny without overt behavioral defects in mating behaviors (FIGS. 15C and 15G). Thus, it was concluded that cil-1 is required for sperm function in both hermaphrodites and males.

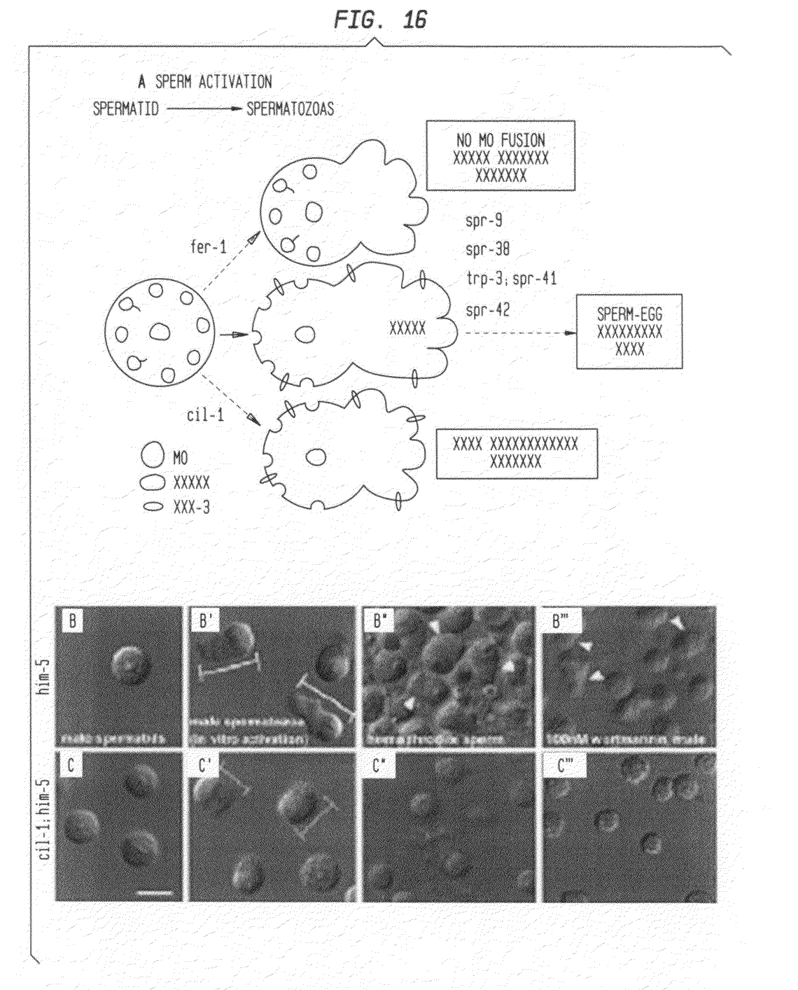

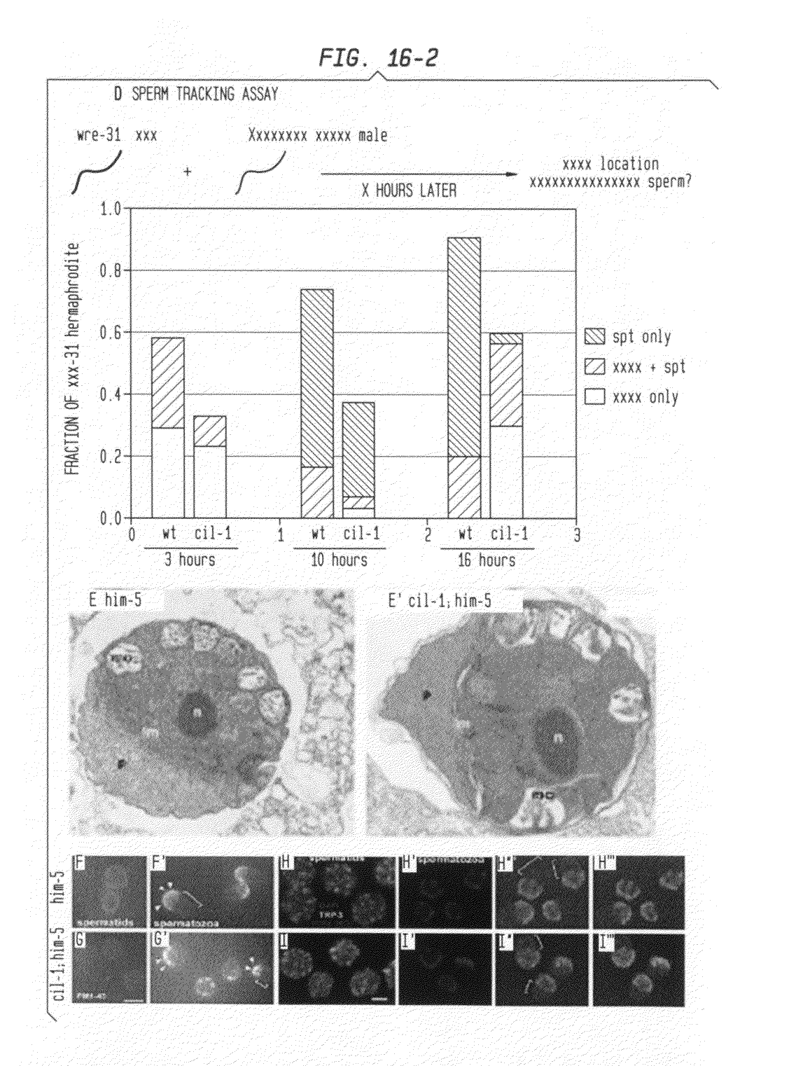

A Spe phenotype may arise from developmental defects in spermatogenesis, sperm activation (spermiogenesis), sperm motility, or sperm-egg interactions. my15 male gonads have normal DAPI staining patterns for each meiotic stage in the gonad and normal number of spermatids (inactive 1N sperm) (data not shown). Thus, cil-1 is not required for early spermatogenesis up to spermatid production. Defective sperm-egg interactions are the basis of Spe phenotypes in spe-9, spe-38, trp-3/spe-41, and spe-42 (FIG. 16). In these Spe mutants, male-derived sperm normally develop, activate, crawl, and compete with endogenous hermaphroditic sperm but cannot fertilize an oocyte. However, my15 male-derived sperm do not compete with endogenous hermaphrodite-derived sperm, as reflected by a large number of self-progeny and extremely low mating efficiency (ME) (1.9%, FIG. 15C). ME of WT males is higher with my15 hermaphrodites (95%) than control hermaphrodites (58%) (FIG. 15C), illustrating that my15 endogenous sperm are nearly incapable of competing with WT male-derived sperm. Hence, cil-1 acts in events between spermatid production and sperm-egg interactions.

(I) cil-1 is Required for Sperm Activation and Motility

During sperm activation, a round spermatid develops into a motile spermatozoon with a pseudopod (FIG. 16A). This process can be mimicked in vitro by chemical activators such as the ionophore monensin or Pronase. Within 15 min of Pronase application, the majority of WT male spermatids (63.4%, n=347) extend pseudopods (FIGS. 16B and 16B′), and the average length of spermatozoa is 8.13±1.11 μm (±standard deviation [SD], n=95) (FIG. 16B′). In contrast, only 47.8% (n=128) of my15 male spermatids develop pseudopods after Pronase activation (FIGS. 16C and 16C′), with a significantly shorter spermatozoa length (6.37±0.61 μm [n=112], p=1.09E-28, FIG. 16C′). The diameter of my15 spermatids is also slightly smaller (6.22±0.54 μm, n=136) than WT (6.73±0.65 μm, n=110, p=8.08E-07). In vivo sperm activation defects were observed also in my15 hermaphrodites. In wild-type, 83.3% of spermatozoa possess pseudopods (n=36, FIG. 16B″), whereas only 8.3% do in my15 mutants (n=72, FIG. 16C″). When present, my15 pseudopods are significantly shorter (average length of longest axes of sperm; 4.32±0.38 μm, n=6) than WT (5.43±0.62 μm, n=37, p=1.14E-4). Thus, cil-1 positively regulates sperm activation in vitro and in vivo.

To measure my15 sperm motility, a time-lapse sperm-tracking assay was performed. WT male sperm crawl from the uterus to the spermatheca over time, with the majority localizing at the spermatheca in 16 hr (FIG. 16D). In contrast, my15 sperm are not observed in the spermatheca at 16 hr despite being present at 4 hr (FIG. 16D). These data suggest that my15 sperm display reduced motility and, consequently, are not retained in the hermaphrodite spermatheca. After in vitro chemical activation with monensin, WT sperm crawl at the rate of 0.33±0.04 μm/s (n=7) on glass slides, whereas isolated my15 sperm are immotile.

Sperm activation involves fusion of membranous organelles (MOs) to the PM, exocytosis, and pseudopod extension. To examine MOs, the MO-specific monoclonal antibody 1CB4 and the lipophilic dye FM1-43 were used. In WT, the majority of MOs are found at the cell periphery of round spermatids (FIG. 16H). After activation, MOs are excluded from the pseudopod (FIG. 16H′). In my15 sperm, 1CB4 staining for MO morphology and localization before and after activation appears to be WT (FIGS. 16I and 16I′). In both WT and my15, FM1-43 labels the PM of the round spermatids (FIGS. 4F and 4G) and, after activation, concentrates at the site of membrane fusion on the spermatozoon cell body (FIG. 16F′ and 16G′, arrowheads). cil-1 is not essential for MO morphology, localization, or fusion.

The ultrastructure of my 15 sperm is largely unaffected as determined by transmission electron microscopy (TEM). The early stages of spermatogenesis in WT and my15 appear very similar (data not shown). Occasionally, dissected males will produce spontaneously activated spermatozoa, and we examined such cells in both WT and my15 (FIGS. 16E and 16E′). In both cases, a pseudopod is extended and separated from the cell body by laminar membranes. MOs successfully fuse with the PM in my15, consistent with immunostaining and MO fusion assay (FIGS. 16G′ and 16I′).

cil-1 may regulate membrane receptor localization in sperm. TRP-3/SPE-41 acts in sperm, translocates from MOs to the PM upon activation, and is required for fertilization. In WT and my15 spermatids, anti-TRP-3 labels cytoplasmic puncta partially overlapping with MOs (FIGS. 16H and 16I). In WT and my15 spermatozoa, TRP-3 is detected ubiquitously on the PM of the pseudopod and cell body (FIGS. 16H″ and 16I″). cil-1 is not required for TRP-3/TRPC translocation to the PM.

### (J) CIL-1 and PI 3-Kinase Activity Antagonistically Regulates Sperm Activation

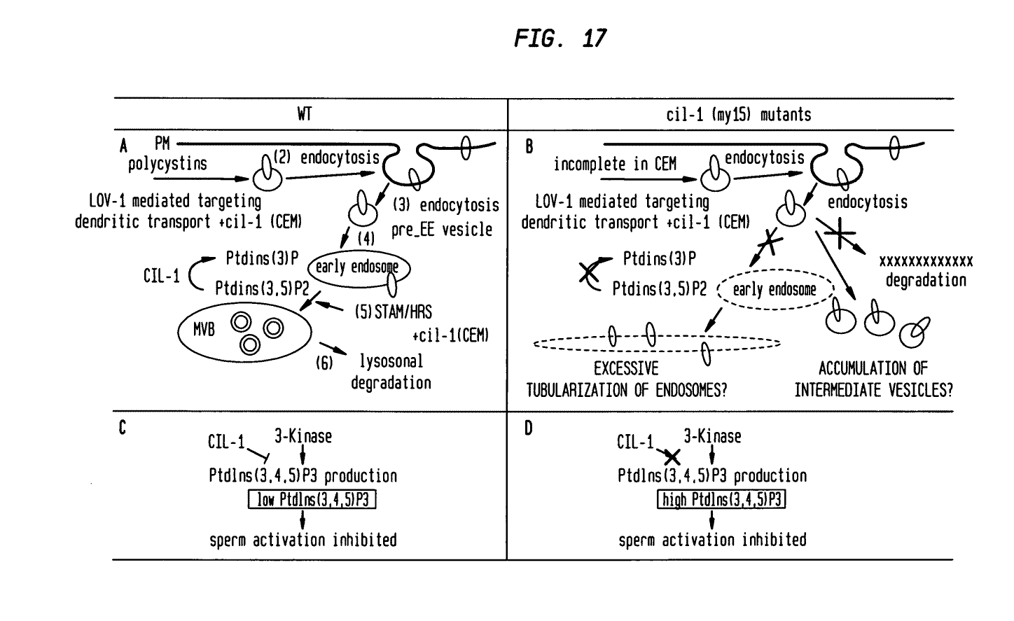

Because PI biosensors show that CIL-1 hydrolyzes PI(3,4,5)P3 and PI(3,5)P2, it was examined whether 3-kinase activity antagonizes CIL-15-phosphatase function. To this end, 1 nM, 10 nM, and 100 nM wortmannin, a pharmacological inhibitor of PI 3-kinases that produce PI(3,4,5)P3 from PI(4,5)P2 [27], were applied to WT and my15 spermatids. At all concentrations tested, wortmannin acts as an in vitro activator of WT but not my 15 spermatids, with 100 nM being the most effective (compare FIG. 16B′with FIG. 16C′). The low dose (1 nM) of wortmannin suggests specificity and indicates that PI 3-kinase activity and cil-1 act antagonistically in sperm activation (FIGS. 17C and 17D).

While the described invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the described invention. All such modifications are intended to be within the scope of the claims appended hereto.

We claim:

1. A method for identifying a therapeutic target for treating a disease comprising a ciliopathy, the method comprising steps:
(a) providing an animal model system of the ciliopathy for testing a putative therapeutic agent, wherein the animal comprises ciliated cells;
(b) labeling the ciliated cells of the animal of step (a) with a traceable agent;
(d) administering a disruptive agent wherein the disruptive agent affects the function of at least one therapeutic target of the animal of step (a),
(e) comparing a measurable trait of the labeled ciliated cells of step (b) with a wild type animal,
(f) identifying the at least one therapeutic target of step (d) as a therapeutic target for treating a ciliopathy,
wherein the therapeutic target affects at least one trait of cilia or a dendrite extension of the ciliated neurons.

2. The method according to claim 1, wherein the animal is Caenorhabditis elegans .

3. The method according to claim 1, wherein administering step (d) further comprises associating the disruptive agent with the at least one therapeutic target.

4. The method according to claim 1, wherein the cell is a neuron.

5. The method according to claim 1, wherein the measurable trait is an increase or decrease of a parameter that modulates at least one function of the therapeutic target.

6. The method according to claim 1, wherein the measurable trait is morphology.

7. The method according to claim 1, wherein the disruptive agent of step (d) is a double stranded RNA molecule.

8. The method according to claim 1, wherein the method further comprises identifying a modulator of the disruptive agent of step (d), wherein the modulator at least partially affects the measurable trait of the therapeutic target.

9. The method according to claim 8, wherein the measurable trait is morphology.

10. The method according to claim 9, wherein the morphology is of cilia.

11. The method according to claim 1, wherein the method further comprises administering an modulator of the disruptive agent of step (d) to a patient suffering from a ciliopathy, wherein the modulator of the disruptive agent at least partially affects the measurable trait of the therapeutic target by increasing the activity of the measurable trait.

12. The method according to claim 1, wherein the method further comprises administering an modulator of the disruptive agent of step (d) to a patient suffering from a ciliopathy, wherein the modulator of the disruptive agent at least partially affects the measurable trait of the therapeutic target by decreasing the activity of the measurable trait.

13. The method according to claim 1, wherein the traceable agent is a lipophilic dye.

14. The method according to claim 1, wherein the traceable agent is According to another embodiment, the traceable agent is a fluorescence protein.

15. The method according to claim 1, wherein the ciliopathy is a cil-1-dependent ciliopathy.

16. The method according to claim 1, wherein the ciliopathy is Joubert Syndrome.

17. The method according to claim 1, wherein the ciliopathy is congenital hepatic fibrosis/Caroli Syndrome.

18. The method according to claim 1, wherein the ciliopathy is an autosomal dominant polycystic kidney disease.

19. The method according to claim 1, wherein the ciliopathy is nephronophthisis.

20. The method according to claim 1, wherein the ciliopathy is Bardet-Biedhl Syndrome.


SEQUENCE LISTING (6 sequences)

SEQ ID NO: 1 - DNA, 23 bp, Unknown
  MAMMALIAN
actcactgctttcaagttgctga

SEQ ID NO: 2 - DNA, 43 bp, Unknown
  MAMMALIAN
agtcgatcgatgcctcgatacctctgttgataagttgagcgag

SEQ ID NO: 3 - DNA, 48 bp, Unknown
  MAMMALIAN
tcgaggcatcgatcgactataacaatggattggaaaataacaatattc

SEQ ID NO: 4 - DNA, 20 bp, Unknown
  MAMMALIAN
tgcaagactattagggagtg

SEQ ID NO: 5 - DNA, 19 bp, Unknown
  MAMMALIAN
aggagccgtacttacatgg

SEQ ID NO: 6 - DNA, 18 bp, Unknown
  MAMMALIAN
aatttgccgcgtcgagac